(19) United States
(12) Patent Application Publication
(10) Pub. No.: US 2003/0138399 A1
(43) Pub. Date: Jul. 24, 2003

(54) Anti-inflammatory therapy for inflammatory mediated infection

(76) Inventors: Peter A. Anton, Los Angeles, CA (US); Janis V. Giorgi, Woodland Hills, CA (US); Julie E. Elliott, Lory Beach, CA (US); Michael A. Poles, Manhattan Beach, CA (US)

Correspondence Address: FISH & RICHARDSON, PC, 4350 LA JOLLA VILLAGE DRIVE, SUITE 500, SAN DIEGO, CA, 92122, US

(21) Appl. No.: 09/569,765

(22) Filed: May 12, 2000

Related U.S. Application Data
(60) Provisional application No. 60/134,091, filed on May 14, 1999.

(51) Int. Cl. C12Q 1/68; A61K 45/00

(52) U.S. Cl. 424/85.1; 435/6

(57) ABSTRACT

Provided are methods for inhibiting the progression of an inflammatory mediated mucosal infection. The methods include administering an effective amount of an anti-inflammatory agent. Also provided are compositions and articles of manufacture for preventing, and inhibiting the activation and progression of a mucosal infection.

### TECHNICAL FIELD

This invention relates the use of anti-inflammatory agents for the treatment of retrovirus infections. Specifically the invention concerns the treatment of HIV infections.

### BACKGROUND

The HIV infection cycle begins with the entry of the virus into the target cell. The human CD4 is believed to be the primary receptor on T cells recognized by HIV. The binding of the HIV envelope glycoprotein (env) to the CD4 receptor results in the fusion of virus and cell membranes, which in turn facilitates virus entry into the host. The eventual expression of env of the surface of the HIV-infected host cell enables this cell to fuse with uninfected CD4-positive cells, thereby spreading the virus. However, HIV can also enter other cells such as monocytes, B cells, and dendritic cells, which can serve as viral reservoirs, even though they may not express CD4. Cytokines are known to affect HIV replication. Pro-inflammatory cytokines promote HIV replication (Fauci, Nature 384:529-534, 1996), while β-chemokines inhibit the replication of obligate CCR5 utilizing viruses (Moore, et al., J. Virol. 70:551-562, 1996), and enhance the replication of CXCR4 utilizing viral isolates (Dolei, et al., AIDS 12:183-190, 1998).

Physical contact between helper T cells and B cells in vivo, mediated by such molecules as CD4, T cell receptor, and MHC class II, is essential in the development of thymus-dependent humoral immunity. The interactions between CD 40 and the CD40 ligand are central to the development and maintenance of immunity. CD40 is a transmembrane glycoprotein of 45 kDa, which is member of a family of surface molecules with homology in their intracellular domain to nerve growth factor receptor, TNF receptors, Fas, CD17, and CD30 (Armitage et al., Nature 357:80-82, 1992). CD40 has been identified on immature and mature B lymphocytes, which when cross-linked by antibodies induces B cell proliferation (Vall et al., Eur. J. Immunol. 19:1464-1467, 1989), on monocytes, dendritic cells, thymic epithelial cells. On antigen presenting cells (APCs) CD40 functions as a costimulatory receptor that promotes antigen-specific T-cell activation (reviewed in Clark et al., Adv. Immunol. 63:43-68, 1996). A ligand for CD40, called gp39, CD40 ligand or CD40L, has also been molecularly characterized (Armitage et al., 1992, supra), and found to be expressed on activated CD4+ Th cells (Spriggs et al., J. Exp. Med. 176:1543-1550, 1992). Cells that express the gp39 protein can trigger B cell proliferation and, through other stimulatory signals, can induce antibody proliferation (Armitage et al., 1992, supra). It has also been reported that engagement of CD40 by CD40L results in enhanced expression of CD80 and CD86, and the secretion of pro-inflammatory cytokines.

The normal intestinal tract is characterized by a low level of mild inflammation, which is fueled by constitutive levels of locally secreted chemokines and cytokines (Shanahan and Anton, Gut Peptides, J. Walsh eds. (Raven Press, Ltd, New York, 1994, page 851; Schreiber et al., Gastroenterology 101:1020 (1991); MacDermott et al., Inflammatory Bowel Diseases 4, 54 (1998); Luster, N.Engl. J. Med. 338:436 (1998)). In healthy controls, gastrointestinal lymphocytes are known to differ functionally and phenotypically from their peripheral blood counterparts (Allison et al., Gastroenterology 99:421 (1990); Jarry et al., Eur. J. Immunol. 20:1097 (1990); McGowan et al., Neuroimmunomodulation 4:70 (1997)). Virtually all mucosal CD4+ lymphocytes express activation markers and are of the CD45RO+ memory subset (Schieferdecker et al., J. Virol. 149:2816 (1992)). In the setting of HIV-1 infection, various phenotypic abnormalities of gut T lymphocytes have been described often associated with depletion of CD4+ lymphocytes (Schnieder et al., Clin. Exp. Immunol. 95:430 (1994)).

Without an effective vaccine, the number of individuals infected with HIV will likely increase substantially. Furthermore, in the absence of effective therapy, most individuals infected with HIV will develop acquired immune deficiency syndrome (AIDS) and succumb to either opportunistic infections and malignancies that result from the deterioration of the immune system, or the direct pathogenic effects of the virus. Despite the present availability of some anti-HIV agents that slow disease progression, a pressing need remains for more effective therapeutics and drug combinations.

### SUMMARY

The present invention is based upon the discovery that retroviruses, such as human immunodeficiency viruses (HIV), provoke an inflammatory state, in contrast to the previous view that HIV is a static or progressive immunodeficient state. Such inflammatory states both provide opportunistic advantages to HIV by providing recruitment of additional inflammatory cells at the site of inflammation bearing receptors used by HIV for infection, as well as activating infected inflammatory cells causing production of viral particles.

In one embodiment, the present invention provides a method for inhibiting an inflammatory mediated infection of mucosal tissue by contacting the tissue with an inhibiting effective amount of an anti-inflammatory agent alone, or in combination with an anti-viral agent. The inflammatory mediated infection can be caused by a virus, such as a retrovirus (e.g., a lentivirus such as an immunodeficiency virus selected from the group consisting of human immunodeficiency virus (HIV) type 1, HIV-type 2, and simian immunodeficiency virus (SIV). The contacting of the tissue with the anti-inflammatory agent alone, or in combination with an anti-viral agent, may be in vivo, in vitro, or ex vivo. The mucosal tissue is typically mammalian and preferably human. Examples of such mucosal tissue include uro-genital tissue, (e.g., vaginal tissue), gastrointestinal tissue, a tissue of the lower GI tract, and nasal-larynx tissue to name a few. The anti-inflammatory agent may be administered locally or systemically, such as by topical administration, intravenous, oral or parenteral administration, respectively. The anti-inflammatory agent, when used in combination with an anti-viral agent, may be administered prior to, simultaneously with or after administration of the anti-viral agent. The anti-inflammatory agent may be any anti-inflammatory agent such as those that cause a decrease in the recruitment of inflammatory cells, a decrease in the production of chemokines, a decrease in the production of pro-inflammatory cytokines, or inhibits the interaction of a chemokine or cytokine receptor with its ligand. Such anti-inflammatory agents may be administered in singlet or combination and also may be administered along with other anti-viral compounds in singlet or combination.

In another embodiment, the present invention provides a method of inhibiting activation of a retrovirus by contacting a cell infected with the virus with a virus-activation inhibiting amount of an anti-inflammatory agent alone, or in combination with an anti-viral agent. Activation of inflammatory cells by autocrine and paracrine effect of pro-inflammatory mediators causes a change in the transcription regulation of inflammatory cells at the site of inflammation. The activation of inflammatory cells through such process in turn results in activation of latent infections of retroviruses. Such retroviruses include lentiviruses such as the immunodeficiency viruses HIV type 1, HIV-type 2, and simian immunodeficiency virus (SIV). The contacting of the cell with the anti-inflammatory agent alone, or in combination with an anti-viral agent, may be in vivo, in vitro, or ex vivo. The anti-inflammatory agent, when used in combination with an anti-viral agent, may be contacted prior to, simultaneously with or after contacting with the anti-viral agent. The cell may be a mucosal cell and is typically mammalian and preferably human. Examples of such mucosal cells include cells derived from uro-genital tissue, (e.g., vaginal tissue), gastro-intestinal tissue, tissue of the lower GI tract, oral-buccal tissue and nasal-larynx tissue to name a few.

In yet another embodiment, the invention provides a method of inhibiting an inflammatory mediated mucosal infection in a subject by contacting the subject with an effective amount of an anti-inflammatory agent alone, or in combination with an anti-viral agent. The inflammatory mediated mucosal infection may be caused by a virus or another pathogen. The virus may be a retrovirus, for example, a lentivirus such as the immunodeficiency virus HIV type 1, HIV-type 2, and simian immunodeficiency virus (SIV). Where the contacting is in vivo, such contacting may be by administering the anti-inflammatory agent locally or systemically, for example by topical administration, intravenous, oral or parenteral administration, respectively. The anti-inflammatory agent, when used in combination with an anti-viral agent, may be administered prior to, simultaneously with or after administration of the anti-viral agent. The subject is typically mammalian and preferably human.

In another embodiment, the invention provides a method of inhibiting transmission of an inflammatory mediated mucosal infection from a subject having or at risk of having an inflammatory mediated mucosal infection to another subject by contacting the subject having or at risk of having the inflammatory mediated mucosal infection with an effective amount of an anti-inflammatory agent alone, or in combination with an anti-viral agent, thereby inhibiting transmission of an inflammatory mediated mucosal infection to the other subject. The inflammatory mediated mucosal infection may be caused by a virus or by another pathogen. The virus may be a retrovirus, for example, a lentivirus such as the immunodeficiency virus HIV type 1, HIV-type 2, and simian immunodeficiency virus (SIV). Where the contacting to the subject having or at risk of having an inflammatory mediated mucosal infection is in vivo, such contacting may be by administering the anti-inflammatory agent locally or systemically, for example by topical administration, intravenous, oral or parenteral administration, respectively. The anti-inflammatory agent, when used in combination with an anti-viral agent, may be administered prior to, simultaneously with or after administration of the anti-viral agent. The subject having or at risk of having an inflammatory mediated mucosal infection is typically mammalian and preferably human.

In still another embodiment, the present invention provides a method of inhibiting progression of an inflammatory mediated infection in a subject by contacting the subject with an effective amount of an anti-inflammatory agent alone, or in combination with an anti-viral agent. The inflammatory mediated mucosal infection may be caused by a virus or another pathogen, or related to infection by the virus or other pathogen. The virus may be a retrovirus, for example, a lentivirus such as the immunodeficiency virus HIV type 1, HIV-type 2, and simian immunodeficiency virus (SIV). Where the contacting is in vivo, such contacting may be by administering the anti-inflammatory agent locally or systemically, for example by topical administration, intravenous, oral or parenteral administration, as respectively. The anti-inflammatory agent, when used in combination with an anti-viral agent, may be administered prior to, simultaneously with or after administration of the anti-viral agent. The subject is typically mammalian and preferably human.

In an additional embodiment, the present invention provides a method for preventing or decreasing the probability of infection of a subject with a human immunodeficiency virus by administering to a subject at risk of an HIV infection a prophylactic effective amount of an anti-inflammatory agent alone, or in combination with an anti-viral agent, which inhibits HIV replication, activation, or progression by reducing the number of inflammatory cells present in any given tissue, such as the uro-genital, gastro-intestinal or other mucosal tissue.

In a further embodiment, the present invention provides a method for preventing or decreasing the probability of human immunodeficiency virus transmission from a subject with an HIV infection to another subject by administering to the infected subject an effective amount of an anti-inflammatory agent alone, or in combination with an anti-viral agent, which inhibits HIV replication, activation, or progression by reducing the number of inflammatory cells present in any given tissue, such as the uro-genital, gastro-intestinal or other mucosal tissue, thereby preventing or decreasing the probability of HIV transmission from the infected subject to another subject.

Also provided is a pharmaceutical composition comprising at least one dose of a therapeutically effective amount of an anti-inflammatory agent, in a pharmaceutically acceptable carrier designed to be delivered to a mucosal tissue, wherein the dose is in an amount effective to inhibit or decrease the probability of immunodeficiency virus progression, infection or transmission.

In another embodiment, the present invention provides an article of manufacture, comprising at least one anti-inflammatory agent and instructions for use of the agent in inhibiting an immunodeficiency virus infection. An article of manufacture can include at least one anti-inflammatory agent alone, or be in a combination with an anti-viral agent. Articles of manufacture include, for example, a condom, sponge, diaphragm, cervical cap, vaginal ring, suppository, and an enema. Instructions for use can be included with an article of manufacture, for example, instructions for use in prophylaxis of immunodeficiency virus infection, or in preventing or inhibiting transmission of immunodeficiency virus from one subject to another.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 shows CCR5 receptor expression on CD4+ lymphocytes from blood and from gut mucosa. Flow cytometry scatter plots (A, B) demonstrate lymphocyte subset analysis to quantify percentages of cells expressing CCR5 and/or CD4 in a representative subject for blood (A) and gut (B). The number on the upper right quadrant of each plot indicates the percentage of CD4+ lymphocytes in that subject that expressed CCR5. (C) The individual data points for the six subjects; data from the blood and gut of each subject. The gut samples of all six subjects had a greater percentage of CCR5+CD4+ cells compared with the blood (P=0.03); differences range from 2.0-5.4-fold.

FIG. 2 shows the number of CCR5 receptors per cell on CD4+lymphocytes from blood and from gut mucosa. Flow cytometry histograms (A,B) demonstrate quantitation of CCR5 expression on CD4+ lymphocytes of one of the six subjects for blood (A) and gut (B). The rightward shift of mean fluorescence index in the CCR5+ CD4+ mucosal cells illustrates the increased numbers of CCR5 receptors per CD4+ lymphocyte. The number above the bars in A and B indicates the number of molecules of CCR5 expressed per CCR5+ CD4+ lymphocyte in the blood and gut of that individual. (C) The gut samples of all six subjects had higher expression of CCR5 compared with the blood (P=0.03); differences range from 1.4 to 3.5-fold. Symbols for each person are the same as those used in FIG. 1.

FIG. 3 shows CXCR4 receptor expression on CD4+ lymphocytes from blood and from gut mucosa. Flow cytometry scatter plots (A,B) demonstrate lymphocyte subset analysis to quantify percentages of cells expressing CXCR4 and/or CD4 in a representative subject for blood (A) and gut (B). The number on the upper right quadrant of each plot indicates the percentage of CD4+ lymphocytes in that subject that expressed CXCR4. (C) The individual data points for the six subjects. Percentages are not different between the two compartments (P=0.3). Symbols for each person are the same as those used in FIG. 1.

FIG. 4 shows the number of picograms of p24 produced by MMC and PBMC after infection with HIVSX or HIVNL4-3. Line graphs indicate the p24 production (picograms of p24 per 104 CD4+ lymphocytes) at 18, 72, and 130 hours after a 3-hour infection with either M-tropic HIVSX (A) or T-tropic HIVNL4-3 (B). After 72 and 130 hours the supernatants from the MMCs cultured in the presence of 20 IU/mL of IL-2 (●) contained greater concentrations of p24 than the supernatants from either PBMC grown with (∘) or without (▾) 20 IU/mL of IL-2. The greater p24 production from the cultured mucosal cells suggests that they are more susceptible than PBMC to replication of M-, or T-tropic HIV-1.

FIG. 5 shows the 3 day/IL-2 culture for isolation of mucosal mononuclear cells yields increased numbers of CD45+, CD3+ CD4+ and CD8+ cells as compared to conventional collagenase/dispase digestion. The mononuclear cell populations isolated by each technique do not appear to differ significantly in their T cell subset make-up.

FIG. 6 shows pre-amplification handling of tissue biopsy samples results in a 5-10% RNA loss. Seronegative samples were ‘spiked’ with 250 copies of the standard LTR sequence pre-extraction (left) and in a parallel sample post-extraction (right). Digitized quantification of 32P emission demonstrated a 5-10% difference between pre and post extraction additions of the same amount of LTR RNA. Standards demonstrate an assay sensitivity of 10 copies.

FIG. 7 shows the internal consistency between samples obtained from different sites at the same circumferential level (30 cm) in the colon. Each sample was run in duplicate. On average, there was a 0.2 log SD between samples from each individual. All subjects had undetectable plasma viral loads.

FIG. 8 shows a quantitative measurement of HIV in rectal biopsies. DNA was extracted from duplicate biopsies of subjects with undetectable plasma HIV RNA. qPCR of the HIV LTR sequence was conducted and actual detected copy numbers from each of the 2 samples from each subject recorded (lower panel). (The 4 different subjects are labeled “Samples #1-4”). β-globin quantitation and standard curves were performed in triplicate on each subject's 2 samples (only one biopsy's results are displayed in the top panel. Calculated numbers of HIV DNA copies are reported per 2×106 β-globin copies (1×106 cells).

FIG. 9 shows that the isolation process does not alter relevant receptor expression. Flow diagrams of peripheral blood mononuclear cells (PBMC) stained directly [upper panels] with antibodies to CD4, CD8, CCR5 or CXCR4 as identified on the horizontal and vertical axes. Lower panels show results of parallel staining of the same individual's PBMC following exposure to the isolation process used for mucosal mononuclear cells.

FIG. 10 shows that the CCR5 receptor is expressed on a significantly greater percentage of mucosal CD4+ T cells compared to blood. Samples from healthy, seronegative controls (n=6) were stained for CD4 and CCR5 (initial gating of mucosal samples: CD45 vs. side scatter). Numbers in the upper right quadrants represent the percentage of CD4 T cells expressing CCR5 receptors.

FIG. 11 shows that CCR5 receptor number per cell is significantly increased on mucosal CD4 T cells compared to blood CD4 T cells in the same subject samples shown in FIG. 10. Histograms demonstrate the marked rightward shift of mean channel fluorescence correlating with increased number of receptors per cell in the mucosal preparation.

FIG. 12 shows increased mucosal compared to blood CCR5 expression on CD4+ T cells are detected in normal, inflammatory, and HIV-infected samples. Mean percentages of CD4+ and CCR5+ double-stained cells are shown from seronegative healthy controls (n=6), inflammatory controls (n=4) and HIV-infected (n=8). P values under the subject's category on the x-axis identify the significance between blood and gut cells within the clinical group. P values at the top of the graph identify significance levels between the CCR5-expressing CD4 T cells in the mucosal compartment between clinical groups.

FIGS. 13A and 13B show that CCR5+ CD4:CD8 ratios in (A) blood and (B) gut decline in IBD and HIV. The left panel shows relative CCR5 expression in blood from healthy, seronegative controls, seronegative inflammatory controls and subjects with stable HIV infection. The changing ratios of CCR5-expressing CD4+ T cells to CCR5-expressing CD8+ T cells are boxed underneath. Similar presentations for mucosal lymphocytes are shown in (A).

FIG. 14 shows that the amount of p24 (indicator of HIV production) is significantly higher in mucosal mononuclear cells (MMC) than in peripheral blood mononuclear cells (PBMC).

FIGS. 15A and 15B show that CD8+ cells are increased in colon from an HIV infected patient. Biopsies from (A) HIV(−) and (B) HIV(+) colon. CD8+ cells are indicated as brown (darkened in black and white photo).

FIGS. 16A and 16B show that CCR5+ cells are increased in colon of an HIV infected patient. Biopsies from (A) HIV(−) and (B) HIV(+) colon. CCR5+ cells are indicated by brown staining (darkened in black and white photo).

FIGS. 17A through 17D show the amount of (A) RANTES; (B) IFNγ; and (C) TNF in uninfected, HIV(+) with low mucosal viral load and HIV(+) with high mucosal viral load. (D) shows the increased amount of CD4 cell activation (as indicated by increased HLA-DR that occurs due to HIV-induced increases in pro-inflammatory cytokines. The y-axis denotes the % of CD4 cells activated and the x-axis denotes viral load.

FIG. 18 shows increased amounts of virus (Nlegfp) produced by mucosal cells (MMC) versus blood cells (PBMC), as indicated by increased expression of green fluorescent protein when Nlegfp is replicated in the cells.

FIG. 19 shows the effect of Asacol (mesalamine) on HIV replication. Cultured cells infected with HIV were treated with 5-ASA or AZT in the amounts indicated. Luciferase expression, which indicates the amount of HIV replication, was quantified using a luminometer. The data shown represent nine separate studies.

### DETAILED DESCRIPTION

It must be noted that as used herein and in the appended claims, the singular forms “a,” “and,” and “the” include the plural unless the context clearly dictates otherwise. Thus, for example, reference to “a cell” includes a plurality of such cells.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices, and materials are now described.

All publications mentioned herein are incorporated herein by reference in full for the purpose of describing and disclosing the cell lines, antibodies, and methodologies, which are described in the publications which might be used in connection with the presently described invention. In case of conflict, the present specification, including definitions, will control. The publications discussed above and throughout the text are provided solely for their disclosure prior to the filing date of the present application. Nothing herein is to be construed as an admission that the inventors are not entitled to antedate such disclosure by virtue of prior invention.

As used herein, the term “inhibit” or “inhibiting” means to reduce the activity, function or property referred to by a measurable amount, such as a reduction of at least 30% or more. Where there are multiple different activities that may be inhibited (for example, preventing cell recruitment, production of pro-inflammatory mediators, cell or viral activation, viral replication, or viral progression/proliferation), the reduction of any single activity (with or without the other activities) is sufficient to fall within the scope of this definition. In addition, where a single or where multiple agents are administered to inhibit activity, the reduction by a single agent of any single activity or the reduction by a combination of agents of any single activity is sufficient to fall within the scope of this definition. An “inflammation inhibiting amount” means that amount of an inflammatory agent necessary to modulate, inhibit, or suppress inflammatory responses or symptoms.

As used herein, the term “activation,” when used in reference to a virus, means an increase in the number of virus particles or viral load systemically or locally, or an increase in synthesis of viral protein or nucleic acid in a cell. The increase is typically induced by a change in a transcriptional state of an infected cell, which results in increased virus production by the cell. The increase may also occur independently of such a change in transcriptional state. Changes in a transcriptional state of an infected cell can be induced by cytokines, chemokines and other molecules that modulate cell proliferation, differentiation or mobilization (chemotaxis). Changes in a transcriptional state of an infected cell can also be induced by infection by the virus or another pathogen (bacterium, fungi, mycobacterium, etc.), or induced by exposure to an immune-modulating antigen (e.g., LPS). Activation, which typically results in increased amounts of virus, in turn can lead to increased numbers of cells infected by the virus, which is referred to as “virus spreading.”

As used herein, the term “inflammatory mediated,” when used in reference to an infection, disease, disorder or condition, means an infection, disease, disorder or condition which progresses or spreads, or accelerates or worsens in response to an inflammatory response of the subject. In the example of a retroviral infection, such as HIV, the progression can occur, for example, through an increase in target (uninfected) cells migrating to the site of inflammation where infected cells and virus are present. The recruited cells provide new targets for viral infection, which results in virus spread and, in turn, progression of the infection. Inflammatory mediated also includes the situation where an infected cell (e.g., in a latent state), when exposed to an immune modulatory molecule or other signaling molecule (e.g., a pro-inflammatory cytokine or other molecule that increases inflammatory response), modulates the cells' transcriptional state, thereby stimulating or increasing production of the virus by the cell.

As used herein, “mucosal tissue” means any tissue in which mucosal cells are found, such tissues, include, for example, gastro-intestinal tissues (e.g., the stomach, small intestine, the large intestine, the rectum), uro-genital tissue (e.g., vaginal tissue, penile tissue, urethra), nasal-larynx tissue (e.g., nasal tissue, larynx tissue), mouth (buccal tissue) to name a few. Other mucosal tissues are known and easily identifiable by one of skill in the art.

The inventors have discovered that retroviral infections (e.g., immunodeficiency viral infection such as HIV infections) are an inflammatory condition present in the tissue of a subject (e.g., mucosal tissue). Most reports have emphasized a state of lymphopenia or “anti-inflammation” in the mucosa paralleling that seen progressively in the blood of infected subjects. Because mucosal tissue is populated by an increased number of activated, memory, co-receptor expressing CD4+ T cells in healthy uninfected individuals, the vulnerability to infection through these tissues is high. In a typical response to infection, mucosal immune cells (most likely CD8+ T-lymphocytes and macrophages) secrete increased levels of pro-inflammatory chemokines and cytokines with the intent of recruiting additional T-lymphocytes to the mucosal infection site. This heightened response or “inflammatory response”, although instigated with the intent of limiting infection, serves to provide significantly increased numbers of potential new targets for infection, favoring spread of the virus. Thus, the present invention relates to the treatment of a wide range of retroviral infections, retroviral related diseases and disorders using any number of anti-inflammatory agents alone, or in combination with any number of antiviral agents, to prevent or inhibit recruitment of additional susceptible cells to the inflamed tissue of a subject, thereby preventing activation of inflammatory cells through inflammatory mechanisms such as activation by cytokines and other pro-inflammatory mediators by inhibiting these pathways as well as preventing transmission to other subjects (uninfected or infected) exposed to the mucosal tissue. The retroviral infections capable of treatment using the methods of the present invention include retroviral disorders caused by a large number of retroviruses.

For example, in SIV-infected macaques, the gastrointestinal tract is the major site of early CD4+ lymphocyte depletion and viral replication to such an extent that it has been suggested that SIV infection may primarily be a disease of the mucosal immune system (Veazey et al., Science 280:427 (1998); MacDonald and Spencer, Gastrointestinal and Hepatic Immunology, R. H. Heatley, Ed. (Cambridge University Press, 1994). In humans, HIV infection also involves the mucosal immune system and infectious viral particles have been recovered directly from mucosal samples and in situ studies have demonstrated that lamina propria T lymphocytes are among the first cells that are infected (Koteler et al., Am. J. Pathol. 139:823 (1991); Heise et al., J. Infect. Dis. 169:1116 (1994); Heise et al., Am. J. Pathol. 142:1759 (1993); Smit-McBride et al., J. Virol. 72:6646 (1998); Clayton et al., Gastroenterology 103:919 (1992); Ellakay et al., Am. J. Clin. Pathol., 87:356 (1987); Jarry et al., Histopathology 16:133 (1990); Lacner et al., Am. J. Pathol. 153:481 (1998). Moreover, the mucosal lining of the rectosigmoid colon is a primary site for viral introduction during anal-insertive intercourse (Patterson et al., Am. J. Pathol. 153:481 (1998)).

Inflammation

Inflammation results from a number of individual and related cascades or reactions caused by pro-inflammatory mediators including cytokines, prostaglandins, leukotrienes, chemokines, adhesion molecules (e.g., LFA-1) and others known to those of skill in the art. For example, chemokine receptors play a pivotal role in permitting viral entry into a CD4+ cell. The ligands for these receptors, called chemokines, are also important. These chemokines, along with pro-inflammatory cytokines are the main stimulators of cells but also play another role in amplification of the inflammatory cascade. These soluble inflammatory mediators are derived mainly from CD8+ T cells. Once produced they can act in a paracrine and autocrine fashion to further activate cells in their vicinity and recruit additional T cells to the site of inflammation. These additional lymphocytes are themselves activated, contributing to the amplifying inflammatory cascade.

Inflammation results in stimulation of lymphocytes and macrophages (CD4+ cells). Those inflammatory cells that harbor HIV when they are stimulated will be induced to produce large amounts of the virus. The intestine, even in healthy HIV-uninfected patients, maintains a state of low-level physiologic inflammation that is necessary to protect the interior milieu from the bath of potential pathogens that contact its surface. The great majority of lymphocytes and macrophages that compose this infiltrate express are therefore stimulated. The primary target of HIV, in which the virus most effectively replicates, is the stimulated CD4+ cell. This type of cell fills the gastrointestinal mucosa. Increased viral replication results in greater spread of HIV throughout the mucosa and higher mucosal HIV viral loads. The predominant aim of anti-HIV therapy is to decrease the ability of HIV to replicate and therefore spread amongst CD4+ cells which are eventually destroyed by the virus. When replication of HIV is effectively reduced so too is to ability of the virus to develop mutations in its genetic material that result in resistance to antiviral medications. Immunosuppressive activity, as used herein, refers to inhibiting or decreasing the ability of B and T cells to react to be recruited or become activated to a site of inflammation. NSAIDs should diminish prostaglandins (PG) synthesis. PGs are cytostatic agents.

Other signals which activate inflammatory cells include binding of an adhesion receptor, for example, LFA-1 (CD11a and CD18), to one of its counter-receptors such as ICAM-1 (CD54) (Staunton et al., 1990, Cell 61:243-254). If the second signal is blocked, the antigen-specific T-cells are induced to die by apoptosis or to enter a state of cellular anergy. Blockage of this interaction by monoclonal antibodies to LFA-1 and ICAM-1 results in increased survival time for mice receiving a heart allograft (Isobe et al., 1992, Science 255: I125-1 127).

The gastrointestinal mucosa is one element of this lymphoid tissue and increasing evidence suggests that HIV involves the mucosa at all stages of disease. Not only is the gastrointestinal tract the route of transmission for the majority of patients, but it is the largest lymphoid organ. As mentioned above, the gastrointestinal mucosa is characterized by a state of low-level physiologic inflammation, and the majority of its lymphocytes are activated. The naturally high concentration of pro-inflammatory cytokines that are present in the mucosa appear to enhance HIV replication in this site, resulting in a high mucosal HIV viral load and successive rounds of infection of new target gastrointestinal CD4+ cells.

The inventors have found that the majority of gastrointestinal CD4+ T cells express the chemokine receptors that are necessary for HIV entry. The vast majority of lymphocytes of the gastrointestinal mucosa express both CCR5 and CXCR4. In the case of CCR5, it also appears that the mucosal mononuclear cells (MMCs) express higher levels of this receptor than blood derived monocytes on a per cell basis. The inventors have found that the mucosal cells are more susceptible to HIV than are peripheral blood cells in vitro.

HIV nucleic acids can be found in the mucosa of the majority of HIV-infected individuals; Kotler et al. detected HIV DNA by PCR using gag-specific primers in 70% of 20 patients he investigated. The inventors have found that even patients with undetectable plasma viral loads have replicating virus in their mucosa. A high SIV viral load is seen in the gastrointestinal mucosa whether the macaque is infected via the gut or via the parenteral route suggesting that the mucosa has a high intrinsic susceptibility to HIV. After infection, these macaques exhibit a profound early (within 7 to 21 days) loss of gastrointestinal mucosal CD4+ cells. This sign of vigorous HIV activity was not mirrored in other lymphoid sites. In humans, mucosal CD4+ cell depletion has been described in the colon and duodenum during both the early asymptomatic phase of chronic infection and after the onset of clinical AIDS.

The inventors have discovered that retroviral infections, for example, infections associated with immunodeficiency viruses, and more specifically HIV are associated with a state of inflammation. The inflammation can be cellular, soluble, or both. The inflammation can be in any number of tissues which are susceptible to infection by a virus (e.g., mucosal tissue). Demonstrations of the inflammatory process related to HIV infection and the role of chemokines and chemokine receptors (which function as co-receptors for HIV) provides for a novel therapy for treating such retroviral infections caused by immunodeficiency-related viruses, for example HIV-1/2.

Inflammatory cells found in regions of mucosal inflammation include a majority of CD 4-positive T-lymphocytes, are of the activated, memory phenotype, express high levels of the requisite co-receptors for HIV and are the preferred target cell for HIV. The co-receptors including the chemokine receptors are a normal part of the endogenous inflammatory immune response functioning as receptors for β-chemokines. These chemokines and their receptors, when activated, trigger a marked recruitment of circulating inflammatory cells to the mucosal site, resulting in cellular and soluble inflammation. Inhibiting mucosal tissue infection, which is enhanced by the presence of inflammation, by providing anti-inflammatory agents will provide an effective method in reducing the activation, progression, and spread of such mucosal tissue diseases.

Accordingly, the use of anti-inflammatory agents pose a useful method for mitigating, controlling, or diminishing the inflammatory response caused by a retroviral infection. Because inflammation usually results in the recruitment and activation of other inflammatory cells through pro-inflammatory mediators, including, but not limited to, prostaglandins, leukotrienes, cytokines, chemokines and others recognized by those of skill in the art, reducing such recruitment and activation will reduce the available number of CD 4-positive cells available for HIV infection.

Anti-inflammatory agents useful in the invention, thus, include agents that decrease the recruitment of inflammatory cells, decrease the production of chemokines, and pro-inflammatory cytokines that foster the perpetuation of the inflammatory cascade, and agents that inhibit chemokine or cytokine receptors (e.g., by inhibiting interaction of a chemokine receptor with its ligand) thereby preventing propagation of the inflammatory message. Such agents include “anti-inflammatory antibodies” which bind to and prevent the biological activity of the protein molecules described above antibody. Such antibodies include antibodies designed to interact with cytokines, cytokine receptors, chemokines, chemokine receptors, which are designed or provided to a subject (e.g., a human) to reduce or prevent an inflammatory response.

The invention also contemplates various pharmaceutical compositions that block retroviral and immunodeficiency virus replication or cytokine secretion in response to an immunodeficiency replication. The pharmaceutical compositions according to the invention are prepared by bringing an antibody, an isolated peptide, a nucleic acid sequence, or other anti-inflammatory agent or drug according to the present invention into a form suitable for administration (e.g., a pharmaceutically acceptable carrier) to a subject using carriers, excipients and additives or auxiliaries. Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol and polyhydric alcohols. Intravenous vehicles include fluid and nutrient replenishers. Preservatives include antimicrobial, anti-oxidants, chelating agents and inert gases. Other pharmaceutically acceptable carriers include aqueous solutions, non-toxic excipients, including salts, preservatives, buffers and the like, as described, for instance, in Remington's Pharmaceutical Sciences, 15th ed. Easton: Mack Publishing Co., 1405-1412, 1461-1487, 1975, and The National Formulary XIV., 14th ed. Washington: American Pharmaceutical Association, 1975, the contents of which are hereby incorporated by reference. The pH and exact concentration of the various components of the pharmaceutical composition are adjusted according to routine skills in the art. See Goodman and Gilman's The Pharmacological Basis for Therapeutics, 7th ed. Such pharmaceutical compositions may include one or more anti-inflammatory agents and one or more anti-viral agents in combination.

In another embodiment, the invention relates to a method of blocking or inhibiting replication or spread of an immunodeficiency virus or the secretion of cytokines in response to an immunodeficiency virus. This method involves administering to a subject a therapeutically effective dose of a pharmaceutical composition containing the compounds of the present invention and a pharmaceutically acceptable carrier. “Administering” the pharmaceutical composition of the present invention may be accomplished by any means known to the skilled artisan. By “subject” is meant any mammal, preferably a human.

The pharmaceutical compositions are preferably prepared and administered in dose units. Solid dose units are tablets, capsules, and suppositories. For treatment of a patient, depending on activity of the compound, manner of administration, nature and severity of the disorder, age, and body weight of the patient, different daily doses are necessary. Under certain circumstances, however, higher or lower daily doses may be appropriate. The administration of the daily dose can be carried out both by single administration in the form of an individual dose unit or else several smaller dose units and also by multiple administration of subdivided doses at specific intervals.

The dosage should not be so large as to cause adverse side effects, such as unwanted cross-reactions, anaphylactic reactions, and the like. Generally, the dosage will vary with the age, condition, sex, and extent of the disease or infection in the patient and can be determined by one skilled in the art. The dosage can be adjusted by the individual physician in the event of any contraindications and can be readily ascertained without resort to undue experimentation. In any event, the effectiveness of treatment can be determined, for example, by monitoring the level of HIV RNA or DNA viral burden in a patient infected with an immunodeficiency virus at the site of inflammation (e.g., mucosal tissue) or by other means including measurement of inflammatory mediators, cytokines, chemokines and/or CD4+ cells. An decrease or stabilization in the relative number of CD4+ cells, level of cytokines, pro-inflammatory mediators or chemokines in the tissue should correlate with the level of inflammation in the individual or tissue.

The pharmaceutical compositions according to the invention are in general administered topically, intravenously, orally or parenterally or as implants. Rectal and vaginal administration may prove more effective as these are typically the sites of first contact and inflammation of mucosal tissue. Such sites can be contacted to prevent or inhibit infection or transmission. Suitable solid or liquid pharmaceutical preparation forms are, for example, granules, powders, tablets, coated tablets, (micro)capsules, suppositories, syrups, emulsions, suspensions, creams, gels, aerosols, drops or injectable solution in ampule form and also preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of present methods for drug delivery, see Langer, Science, 249:1527-1533, 1990, which is incorporated herein by reference.

The pharmaceutical compositions according to the invention may be administered locally or systemically. By “therapeutically effective dose” is meant the quantity of a compound according to the invention necessary to prevent, to cure or at least partially arrest or decrease the symptoms of the disease and its complications. Amounts effective for this use will, of course, depend on the severity of the disease or infection and the weight and general state of the subject. Typically, dosages used in vitro may provide useful guidance in the amounts useful for in situ administration of the pharmaceutical composition, and animal models may be used to determine effective dosages for treatment of particular disorders. Various considerations are described, e.g., in Gilman et al., eds., Goodman and Gilman's: the Pharmacological Bases of Therapeutics, 8th ed., Pergamon Press, 1990; and Remington's Pharmaceutical Sciences, 17th ed., Mack Publishing Co., Easton, Pa., 1990, each of which is herein incorporated by reference. Effectiveness of the dosage can by monitored by determining, for example, the level of HIV RNA or DNA or viral burden in a patient infected with an immunodeficiency virus at the site of inflammation (e.g., mucosal tissue), by a decrease in one or more symptoms associated with the infection, or by other means including measurement of amounts of inflammatory mediators, cytokines, chemokines and/or CD4+ cells, using methods well known to one of ordinary skill in the art.

The immunotherapeutic method of the invention includes a prophylactic method directed to those hosts at risk for the immunodeficiency virus infection. For example, the method is useful for humans at risk for HIV infection. A “prophylactically effective” amount of an anti-inflammatory agent, for example, refers to that amount which is capable of inhibiting HIV replication, activation, or progression by reducing, inhibiting or preventing an inflammatory response. Transmission of HIV occurs by at least three known routes: sexual contact, blood (or blood product) transfusion and via the placenta. Infection via blood includes transmission among intravenous drug users. Since contact with HIV does not necessarily result in symptomatic infection, as determined by seroconversion, all humans may be potentially at risk and, therefore, should be considered for prophylactic treatment by the therapeutic method of the invention.

Anti-inflammatory compositions useful in the invention for prophylactic activity or for preventing transmission include, for example, condoms coated or lubricated with an anti-inflammatory agent, condoms comprising a capsule present on the condom that upon use ruptures to release an anti-inflammatory agent, vaginal products including diaphragms, cervical caps, sponges, and rings coated or lubricated with an anti-inflammatory agent; vaginal douches, creams, gel lubricants, suppositories, foams, or spermicidal gels which also contain an anti-inflammatory agent, and other compositions and articles of manufacture known to those of skill in the art for local delivery of an anti-inflammatory agent to a mucosal tissue contacted during sexual intercourse. Such articles of manufacture are useful in preventing or inhibiting infection of a subject by another, or useful in preventing or inhibiting transmission from an infected subject to another.

The compositions described herein and useful in a method of the invention can be administered to a patient prior to infection with an immunodeficiency virus (i.e., prophylactically) or at any of the stages described below, after initial infection, or after infection in order to prevent subsequent transmission. For example, HIV infection may run any of the following courses:

(1) Approximately 15% of infected individuals have an acute illness, characterized by fever, rash, and enlarged lymph nodes and meningitis within six weeks of contact with HIV. Following this acute infection, these individuals become asymptomatic.

(2) The remaining individuals with HIV infection are not symptomatic for years.

(3) Some individuals develop persistent generalized lymphadenopathy (PGL), characterized by swollen lymph nodes in the neck, groin and axilla. Five to ten percent of individuals with PGL revert to an asymptomatic state.

(4) Any of these individuals may develop AIDS-related complex (ARC); patients with ARC do not revert to an asymptomatic state.

(5) Individuals with ARC and PGL, as well as asymptomatic individuals, eventually (months to years later) develop AIDS which inexorably leads to death.

Anti-Inflammatory Agents

As used herein, “anti-inflammatory agent” means an agent capable of reducing, preventing or modulating an inflammatory reaction, for example, by decreasing recruitment of inflammatory cells, decreasing chemokine production, decreasing pro-inflammatory chemokine production, decreasing pro-inflammatory cytokine production, or inhibiting interaction of a chemokine or cytokine with its receptor. Such agents include, for example, anti-inflammatory antibodies (e.g., anti-cytokine, anti-receptor antibodies), peptides (e.g., agonist or antagonist of inflammatory mediators, cytokines such as IL-1, or receptors such as IL-1 receptor antagonists or soluble TNF receptors), nucleic acids (e.g., nucleic acids which encode anti-inflammatory agents such as anti-inflammatory peptides, ribozymes or antisense molecules), steroids (e.g., prednisone), non-steroidal anti-inflammatory drugs (e.g., aspirin), a 5-ASA product, commonly used anti-inflammatory drugs and combinations thereof.

Examples of anti-inflammatory antibodies useful in the present invention include antibodies to cytokines and their receptors, such as anti-interleukin receptors, anti-cytokine antibodies (e.g. anti-TNF antibodies, such as REMICADE® made by Centocor), anti-chemokine antibodies (see for example Olson et al., J. of Virol. 73(5):4145-4155 (1999), the disclosure of which is incorporated herein), anti-chemokine receptor antibodies (e.g., anti-CCR5 or anti-CXCR4 receptor antibodies) and combinations thereof. Other antibodies include antibodies to enzymes of enzymatic pathways which produce pro-inflammatory mediators, for example antibodies to type-1 phospholipase A2 as disclosed in U.S. Pat. No. 5,767,249, the disclosure of which is incorporated herein by reference.

Examples of anti-inflammatory nucleic acids useful in the present invention include nucleic acids encoding an anti-inflammatory peptide, a ribozyme that cleaves RNA-encoding pro-inflammatory polypeptides (e.g., cytokines or chemokines), antisense molecules capable of hybridizing to nucleic acid sequence which encode pro-inflammatory mediators, such as cytokines, cytokine receptors, chemokines, chemokine receptors, other inflammatory peptides or receptors as disclosed herein, and combinations thereof, or easily identifiable to one of ordinary skill in the art.

Examples of anti-inflammatory peptides include, for example, LFA adhesion molecule antagonist, cytokine receptor antagonist, transcription factor, soluble TNF-α receptor polypeptide. Other anti-inflammatory peptides include for example, transcription factors such as NF-kappa B (Schottelius A J et al., Int J Colorectal Dis February 1999;14(1):18-28), peptide to platelet factor 4 (U.S. Pat. No. 5,776,892, which is incorporated herein), and peptides based on CD14 as disclosed in U.S. Pat. No. 5,766,593 (the disclosure of which is incorporated herein).

Examples of anti-inflammatory cytokines include, for example, cytokines and transcription factors. Anti-inflammatory cytokines include, for example, IL-13 (Watson M L, Am J Respir Cell Mol Biol May 1, 1999; 20(5):1007-1012), IL-4 and IL-10 (Jarvelainen H A et al., Hepatology May 1999;29(5):1503-10), IL-16 (Klimiuk P A, et al., J Immunol. Apr. 1, 1999;162(7):4293-4299) and other anti-inflammatory cytokines known to those of skill in the art.

Examples of anti-inflammatory agents useful in the present invention include agents from a wide variety of steroidal, non-steroidal, and salicylate water-soluble and water-insoluble drugs and their acid addition or metallic salts. Both organic and inorganic salts may be used provided the anti-inflammatory agent maintains its medicament value. The anti-inflammatory agents may be selected from a wide range of therapeutic agents and mixtures of therapeutic agents which may be administered in sustained release or prolonged action form.

Non-steroidal anti-inflammatory agents (NSAIDs) include numerous compounds of diverse chemical structure. Most if not all are believed to share a common mechanism of action, and almost all are weak organic acids. This large group of compounds can be divided into two main groups, carboxylic acids (R—COOH) and enolic acids (R—COH). Further subdivisions based on chemical structure can be made. The main groups of enolic acids are the pyrazolones, such as phenylbutazone, oxyphenbutazone, dipyrone and isopyrin, and the xicams, which include piroxicam and miloxicam. Carboxylic acid subgroups comprise the salicylates, e.g. acetylsalicylate (aspirin); propionic acids, e.g. ibuprofen and naproxen; anthranilic acids, e.g. meclofenamic acid; phenylacetic acids, e.g. acetaminophen; aminonicotinic acids, e.g. flunixin; and indolines, e.g., indomethacin.

For those NSAIDs for which the mechanism of action is known, most have been found to inhibit the formation of arachidonic acid metabolites through suppression of cyclooxygenase and lipoxygenase pathways and thus lead to a reduction in inflammation mediated by these metabolites. Cyclooxygenase converts arachidonic acid to the cyclic endoperoxides, PGG2 and PGH2 (known as PGs). By action of further specific enzymes, these compounds are converted to different members of the family of inflammatory mediators, the eicosanoids, which includes PGE2 and PGI2. However, the structure of cyclooxygenases varies among tissues, and NSAIDs differ in their ability to combine with each of these enzymes, which explains differences in potency and species responses.

Non-limiting illustrative specific examples of non-steroidal anti-inflammatory agents with brand names, generic names, standard sizes of doses, and chemical structures, include the following medicaments: ibuprofen (e.g., MOTRIN® 300, 400, 600, 800 mg, ADVIL® 200 mg) (±)-2-(p-isobutylphenyl) propionic acid; tolmetin (TOLECTIN®) 5-(p-toluoyl)-1-methylpyrrole-2-acetic acid; naproxen (e.g., ALEVE®, ANAPREX®, or NAPROSYN®) 250, 375, and 500 mg, 6-methoxy-α-methyl-2-naphthaleneacetic acid, (+); flurbiprofen (ANSAID®), 50 and 100 mg, 2-fluoro-α-methyl-[1,1′-biphenyl]-4-acetic acid, (±); sulindac (CLINORN®)150 and 250 mg, 5-fluoro-2-methyl-1-[[p-(methylsulfinyl)phenyl]-methylene]-1H-indene-3-acetic acid; diflunisal (FLOVACIL®) 250 and 500 mg, 2′4′-difluoro-4-hydroxy-[1,1′-biphenyl]-3-carboxylic acid; piroxicam (FELDENE®) 4-hydroxy-2-methyl-N-2-pyridinyl-2H-1,2-benzothiazine-3-carboxamide 1,1-dioxide; indomethacin (INDOCIN®), 25 and 50 mg, 1-(4-chlorobenzoyl)-5-methoxy-2-methyl-1H-indole-3-acetic acid; etodolac (ULTRADOL®) 1,8-deethyl-1,3,4,9-tetrahydropyrano-[3,4-b]indole-1-acetic acid; meclofenamate sodium (MECLOMEN®), 50 and 100 mg, N-(2,6-dichloro-M-tolyl) anthranilic acid, sodium salt, monohydrate; fenoprofen and fenoprofen calcium (NALFON®) as dihydrate, 200 mg and 300 mg, derivative of arylacetic acid, α-methyl-3-phenoxybenzeneacetic acid; ketoprofen (ORUDIS®), 25, 50, and 75 mg, 2-(3-benzoylphenyl)-propionic acid; meclofanamate sodium; mefenamic acid (BONABOL®) N-(2,3-xylyl) anthranilic acid; nabumetone; ketorolac tromethamine; diclofenac sodium (PROPHENATIN®) 2-[(2,6-dichlorophenyl)amino] benzeneacetic acid monosodium salt; bromfenac sodium; phenylbutazone (BUTAZOLIDIN®), 100 mg, 4-butyl-1,2-diphenyl-3,5-pyrazolidinedione; other COX-2 inhibitors such as celocoxib, meloxicam, nimesulide and rofecoxib; suprofen; fenbuprofen; fluprofen; Midol-PMS, acetaminophen, 500 mg; Tylenol Extra Strength, acetaminophen, 500 mg; thalidomide; oxaprozin; salicylate containing compounds and evening primrose oil (containing about 72% linoleic acid and about 9% gamma-linolenic acid), single isomers thereof and combinations thereof.

Non-limiting illustrative specific examples of salicylate anti-inflammatory agents include the following medicaments: bismuth subsalicylate; salsalate; salicylic acid and salicylic acid derivatives, for example, sodium thiosalicylate, choline salicylate, magnesium salicylate, diflunisal, ibuprofen, naproxen, sulindac, diflunisal, salicylsalicylic acid, choline magnesium trisalicylate, acetylsalicylic acid, salsalate, sodium salicylate and combinations thereof; 5-aminosalicylic acid (5-ASA) and 5-ASA containing product or compounds, for example: oral mesalamine (ASACOL®, made by Procter & Gamble Pharmaceuticals; PENTASA® made by Roberts Pharmaceuticals), mesalamine rectal enema or foam or suppository, sulfasalazine, balsalazide, ipsalazide, and olsalazine (DEPENTUM®, made by Pharmacia Upjohn), and mixtures thereof.

Steroidal anti-inflammatory agents include glucocorticoids. Non-limiting illustrative specific examples of steroidal anti-inflammatory agents include the following medicaments: flunisolide, triamcinoline, triamcinoline acetonide, beclomethasone diproprionate, betamethasone diproprionate, hydrocortisone, cortisone, dexamethasone, budesonide, prednisone, methyl prednisolone, prednisolone, esters of any of these compounds and combinations thereof. Other non-limiting examples of anti-inflammatory agents include Thalidomide (made by Celgene).

Retroviruses

Retroviruses are RNA viruses wherein the viral genome is RNA. When a host cell is infected with a retrovirus, the genomic RNA is reverse transcribed into a DNA intermediate which is integrated very efficiently into the chromosomal DNA of infected cells. The integrated DNA intermediate is referred to as a provirus. The family Retroviridae are enveloped single-stranded RNA viruses typically infect mammals, such as, for example, bovines, monkeys, sheep, and humans. Retroviruses are unique among RNA viruses in that their multiplication involves the synthesis of a DNA copy of the RNA which is then integrated into the genome of the infected cell.

The Retroviridae family consists of three groups: the spumaviruses (or foamy viruses) such as the human foamy virus (HFV); the lentiviruses, as well as visna virus of sheep; and the oncoviruses (although not all viruses within this group are oncogenic). The term “lentivirus” is used in its conventional sense to describe a genus of viruses containing reverse transcriptase. The lentiviruses include the “immunodeficiency viruses” which include human immunodeficiency virus (HIV) type 1 and type 2 (HIV-1 and HIV-2) and simian immunodeficiency virus (SIV). In the absence of effective therapy, most individuals infected with a human immunodeficiency virus develop acquired immune deficiency syndrome (AIDS) and succumb to either opportunistic infections and malignancies resulting from either the deterioration of the immune system or the direct effects of the virus. The oncoviruses are further subdivided into groups A, B, C and D on the basis of particle morphology, as seen under the electron microscope during viral maturation. A-type particles represent the immature particles of the B- and D-type viruses seen in the cytoplasm of infected cells. These particles are not infectious. B-type particles bud as mature virions from the plasma membrane by the enveloping of intracytoplasmic A-type particles. At the membrane they possess a toroidal core of ˜75 nm, from which long glycoprotein spikes project. After budding, B-type particles contain an eccentrically located, electron-dense core. The prototype B-type virus is mouse mammary tumor virus (MMTV). No intracytoplasmic particles can be observed in cells infected by C-type viruses. Instead, mature particles bud directly from the cell surface via a crescent ‘C’-shaped condensation which then closes on itself and is enclosed by the plasma membrane. Envelope glycoprotein spikes may be visible, along with a uniformly electron-dense core. Budding may occur from the surface plasma membrane or directly into intracellular vacuoles. The C-type viruses are the most commonly studied and include many of the avian and murine leukemia viruses. Bovine leukemia virus (BLV), and the human T-cell leukemia viruses types I and II (HTLV-I/II) are similarly classified as C-type particles because of the morphology of their budding from the cell surface. However, they also have a regular hexagonal morphology and more complex genome structures than the prototypic C-type viruses such as the murine leukemia viruses (MLV). D-type particles resemble B-type particles in that they show as ring-like structures in the infected cell cytoplasm, which bud from the cell surface, but the virions incorporate short surface glycoprotein spikes. The electron-dense cores are also eccentrically located within the particles. Mason Pfizer monkey virus (MPMV) is the prototype D-type virus.

Retroviruses are defined by the way in which they replicate their genetic material. During replication the RNA is converted into DNA. Following infection of the cell a double-stranded molecule of DNA is generated from the two molecules of RNA which are carried in the viral particle by the molecular process known as reverse transcription. The DNA form becomes covalently integrated in the host cell genome as a provirus, from which viral RNAs are expressed with the aid of cellular and/or viral factors. The expressed viral RNAs are packaged into particles and released as infectious virions.

The retrovirus particle is composed of two identical RNA molecules. Each genome is a positive sense, single-stranded RNA molecule, which is capped at the 5 end and polyadenylated at the 3′ tail. The diploid virus particle contains the two RNA strands complexed with gag proteins, viral enzymes (pol gene products) and host tRNA molecules within a ‘core’ structure of gag proteins. Surrounding and protecting this capsid is a lipid bilayer, derived from host cell membranes and containing viral envelope proteins. The env proteins bind to the cellular receptor for the virus and the particle typically enters the host cell via receptor-mediated endocytosis and/or membrane fusion.

After the outer envelope is shed, the viral RNA is copied into DNA by reverse transcription. This is catalyzed by the reverse transcriptase enzyme encoded by the pol region and uses the host cell tRNA packaged into the virion as a primer for DNA synthesis. In this way the RNA genome is converted into DNA genome.

The double-stranded linear DNA produced by reverse transcription may, or may not, have to be circularized in the nucleus before integration into the host cell genome. The provirus now has two identical repeats at either end, known as the long terminal repeats (LTR). The junction between the two joined LTR sequences produces the site recognized by a pol product—the integrase protein—which catalyzes integration, such that the provirus is always joined to host DNA two base pairs (bp) from the ends of the LTRs. A duplication of cellular sequences is seen at the ends of both LTRs, reminiscent of the integration pattern of transposable genetic elements. Integration is thought to occur essentially at random within the target cell genome.

Transcription, RNA splicing and translation of the integrated viral DNA is mediated by host cell proteins. Variously spliced transcripts are generated. In the case of the human retroviruses HIV-1/2 and HTLV-I/II viral proteins are also used to regulate gene expression. The interplay between cellular and viral factors is important in the control of virus latency and the temporal sequence in which viral genes are expressed.

Retroviruses can be transmitted horizontally and vertically. Efficient infectious transmission of retroviruses requires the expression on the target cell of receptors, which specifically recognize the viral envelope proteins, although viruses may use receptor-independent, nonspecific routes of entry at lesser efficiency. In addition, the target cell type must be able to support all stages of the replication cycle after virus has bound and penetrated (nucleic acid of the virus has entered the cell). Vertical transmission occurs when the viral genome becomes integrated in the germ line of the host. The provirus will then be passed from generation to generation as though it were a cellular gene. Hence, endogenous proviruses become established which frequently lie latent, but which can become activated when the host is exposed to appropriate agents. The antiviral agents for use in the compositions and methods of the invention can target any stage of the virus life cycle.

The oncoviruses (often called the RNA tumor viruses) have been subdivided into two groups of pathogens, namely the acutely transforming and slow transforming retroviruses.

Acutely transforming retroviruses can transform cultured cells and can cause disease rapidly in susceptible animals. These viruses usually carry an oncogene (v-onc) within the viral genome, which is directly responsible for their tumorigenicity, and which is different in each type of virus. The viral oncogenes have been derived from cellular genes that the viruses have acquired, probably as a result of the inclusion of cellular RNA within a viral particle. Subsequent recombination between viral and cellular RNA during reverse transcription leads to the incorporation of the cellular sequences into the viral genome and delivery of this novel unit into the host cell DNA. If the transduced gene normally has a central role in control of cellular growth and differentiation, the changes in coding sequence and/or control of expression that it undergoes on incorporation into the viral genome can render it oncogenic. Such cellular proto-oncogenes (c-onc) may become oncogenic by being placed under novel, virally determined transcriptional control (both quantitatively and temporally), and/or by sustaining critical mutations to the coding sequence. However, full cellular transformation usually requires the expression of v-onc in conjunction with other genetic and epigenetic changes within the target cell.

The slow transforming retroviruses typically do not contain a ‘classical’ oncogene. The mechanism of transformation is believed rather to involve the insertion of provirus near, or in, the coding region of a cellular proto-oncogene, called insertional mutagenesis. The strong promoter and enhancer sequences within the viral LTRs can exert transcriptional effects from distances of up to several kilobase pairs from the proto-oncogene. The normal regulation of expression of the cellular gene is disrupted, and over-expression or inappropriately timed expression can contribute to transformation.

HTLV-I is a slow-transforming virus, causally associated with adult T-cell leukemia (ATL), but it probably promotes T-cell transformation by a different pathway involving virally encoded regulatory proteins, especially p40tax, which transactivate expression of cellular proto-oncogenes. HIV-1 and 2 have also been implicated in both the direct and indirect promotion of various types of malignancy (such as Kaposi's sarcoma), which present much more frequently in AIDS patients than in the general population. However, the direct role of HIV in malignant transformation remains doubtful as many patients who are immunosuppressed as a result of other infections or treatments (e.g. transplant recipients) also develop tumors at increased rates.

The D-type viruses are not etiologically associated with malignancy, although MPMV was initially associated with a mammary tumor in a rhesus monkey. D-type viruses cause immune suppression in simian primates but by an unknown mechanism. Immune suppression is also a feature of infection by the lentiviruses (e.g. HIV and SIV) and variant strains of feline leukemia virus (FeLV). n infection with HIV and FeLV large amounts of unintegrated proviral DNA have been observed, which may be related to the pathogenesis.

The lentiviruses, including HIV-1/2 and visna virus of sheep, are associated with slow progressive disease leading to immune suppression and neurological disorders. HIV is the widely recognized causative agent of the acquired immunodeficiency disease syndrome (AIDS).

The pharmaceutical compositions and methods of the invention, including anti-inflammatory agents (antibodies, peptides, peptidomimetics, chemical compositions, etc.) alone, or in combination with antivirals (antibodies, peptides, virus protein/enzyme inhibitors, etc.) are all useful for treating subjects either having or at risk of having an immunodeficiency virus (e.g., HIV) related disorder, or having or at risk of transmitting an HIV related disorder. AIDS and ARC are particular examples of such disorders. HIV-associated disorders have been recognized primarily in “at risk” groups, including homosexually active males, intravenous drug users, recipients of blood or blood products, and certain populations from Central Africa and the Caribbean. The syndrome has also been recognized in heterosexual partners of individuals in all “at risk” groups and in infants of affected mothers.

Retroviruses have been linked to a wide range of diseases, including anemia, neurological disorders, immune suppression, and malignancy. HTLV-I, for example, is associated with tropical spastic paraparesis, a condition similar in some respects to multiple sclerosis.

Antiviral Agents

As used herein, “antiviral agent” means an agent capable of inhibiting, reducing, preventing or modulating viral infection or production at any step or stage of the viral life cycle, for example, by inhibiting, reducing, preventing an initial step of virus infection, such as virus fusion to a cell through a cell surface receptor or independent of a cell surface receptor; subsequent entry of viral nucleic acid into the cell (“entry inhibitor”); reverse transcription of viral nucleic acid (“reverse transcriptase inhibitor”); integration of reverse transcribed viral nucleic acid into the genome of the cell (“integrase inhibitor”); proviral nucleic acid transcription or replication; translation or formation of mature viral proteins; formation/assembly of infectious viral particles; decreasing budding or release of mature virions from a cell; and decreasing activity or an amount of an enzyme associated with viral fusion or infection, replication, maturation, or budding or release from a cell.

Examples of anti-viral agents include polypeptides or functional mimetics, for example, a soluble cell surface receptor peptide that binds virus, a ligand to the cell surface receptors, an antibody or an antibody fragment that binds to the cell surface receptor or the virus particle thereby preventing binding between the virus and a cell surface receptor present on a cell. Viral proteins particularly attractive for anti-viral agent targeting include envelope polypeptide, gp120 or gp41. Cell surface receptors particularly attractive for anti-viral agent targeting include X4 and R5 receptors.

As used herein, the term “functional mimetic” means a molecule that is chemically or structurally modified, but has one or more activities (i.e., functions), or even increased activity, of the unmodified molecules. Thus, in the case of a functional mimetic of an antiviral agent, the mimetic would retain one or more antiviral activities of the antiviral agent. A particular type of functional mimetic is a polypeptide or peptide mimetic (“peptidomimetic”) which is a compound that mimics the three-dimensional structure of the peptide from which the mimetic is intended to mimic. Peptidomimetic design can be aided through three-dimensional computer modeling techniques and can be designed to have additional characteristics that enhance therapeutic application. For example, antisense molecules can be designed with non-natural nucleotides or chemically modified in order to inhibit nuclease digestion of the antisense in vivo.

Additional examples of antiviral agents are where cellular or viral enzymes important for the viral life cycle, such as viral protease, reverse transcriptase or integrase, are inhibited. Particular examples of anti-viral agents include, for example, viral fusion inhibitors, e.g., T20 and T20 analogues (Trimeris, Inc.); entry inhibitors; integrase inhibitors; protease inhibitors (e.g., saquinavir, ritonavir, indinavir, nelfinavir, amprenavir); a nucleoside reverse transcriptase inhibitor (e.g., zidovudine (AZT), stavudine (d4T), larnivudine (3TC), didanosine (DDI), zalcitabine (ddC), abacavir); a non-nucleoside reverse transcriptase inhibitor (e.g., nevirapine, delavirdine, efavirenz); viral maturation into infectious virus (e.g., “zinc finger injectors,” a class of inhibitors that inhibit proper viral α nuclear capsid protein assembly thereby preventing formation of infectious viral particles); and mixtures thereof. Viral budding or release from a cell can be inhibited by agents that inhibit virus maturation or viral protein maturation.

Control of Mucosal Inflammation

The use of highly active anti-retroviral therapy (HAART) by the majority of HIV-infected patients has resulted in prolonged life expectancy of HIV-infected patients. Unfortunately, despite a reduction of plasma load to undetectable levels, multiple studies have shown continued HIV replication in lymphoid organs. Studies have shown that 88% of patients with undetectable plasma viral load have quantifiable HIV nucleic acid in their gastrointestinal mucosa.

Thus, while HAART may reduce viral load to undetectable levels in the plasma, the vast majority of patients will suffer a rebound increase in plasma viremia when therapy is halted, presumably due to the reservoirs of virus that exist beyond the bloodstream. This viral reservoir has been discovered in lymphoid tissue where the majority of the body's lymphocytes reside (98%). Since the gastrointestinal mucosa contains the majority of the body's lymphocytes (40-65%), it likely represents the largest reservoir of HIV, and, therefore, is a primary target for anti-HIV therapy. Given the increased inflammatory state of the mucosa in HIV disease, one focus of this invention entails methods to decrease mucosal inflammation. Therapies that decrease the levels of pro-inflammatory cytokines and chemokines in the mucosal compartment prevents enhanced replication and perhaps viral development of resistance mutations. In addition, decreased soluble inflammation would also attenuate the recruitment of new CD4+ cells into the mucosa for HIV to infect thereby preventing or inhibiting the lateral spread of HIV among the body's lymphocyte pool.

Identifying Effective Anti-Inflammatory Agents

A number of techniques known to those of skill in the art can be used to assess in vitro, as well as in vivo, the effect of anti-inflammatory agents on the ability to inhibit, treat, or reduce the activation or progression of retroviral infections in mucosal cells, tissues, and subjects. Such techniques are applicable to a wide range of anti-inflammatory agents and mucosal cell infections. For example, using the techniques described below, one skilled in the art would be able to assess the effect of an anti-inflammatory agent on HIV infections in mucosal tissues and mucosal tissue in a subject. For exemplification only, outlined below is a method of assessing the effect of Thalidomide and 5-ASA containing compounds or products, however, such examples are meant to illustrate and not limit the scope of the present invention. Data are also described showing inhibition of HIV replication in cells using mesalamine (Asacol).

Thalidomide

Thalidomide is a potent anti-inflammatory medication. While it owes much of its immunomodulatory effect to its ability to down-regulate TNFα production, it appears to affect multiple sites in the inflammatory cascade. Other putative anti-inflammatory mechanisms include a role in attenuating T cell proliferation, down-regulating the level of activation of lymphocytes, inhibition of lymphocyte chemotaxis, and altering the levels of pro-inflammatory cytokines. Thalidomide may prove to be a beneficial component of HIV therapy by decreasing HIV replication systemically and in the gastrointestinal mucosa. In this regard, thalidomide can be used as an adjunct to HAART in patients with undetectable plasma viral load.

Treatment with thalidomide will result in a decrease in the ability of HIV to replicate in a lymphoid environment that favors HIV replication as evidenced by lower tissue levels of HIV RNA burden in treated subjects.

Studies of the effect of thalidomide on the infectivity of cells by HIV are performed in vitro. Patients with moderate to severely active Crohn's disease (colitis) who have begun therapy with thalidomide will be identified and recruited to help differentiate non-specific inflammatory reactions from infectious effects. Mucosal and blood cells from these subjects will be used to evaluate thalidomide's ability to decrease infectibility, in vitro.

The state of inflammation is enhanced in the presence of HIV infection and is characterized by elevated levels of pro-inflammatory cytokines and chemokines. These soluble mediators play a vital role in chemoattraction of chemokine-bearing CD4+ cells to the mucosa. These targets for HIV are further activated in the mucosal environment by the soluble mediators leading to enhanced HIV replication. To further define the inflammatory state of the mucosa, a number of pro-inflammatory cytokines (TNFα, IL-1, IL-6, γ-IFN) and chemokines (MIP 1a, MIP 1b, RANTES) will be measured.

HIV seropositive patients with negative plasma viral loads, but positive mucosal viral loads will be recruited. These subjects will have stable disease, CD4 cell counts greater than 250 and be without gross mucosal inflammation or gastrointestinal symptoms. The subjects will be seen for a baseline endoscopic evaluation. Endoscopic biopsies from a standardized level (30 cm) will be obtained from these patients. Mucosal biopsies will be used for mucosal mononuclear cell (MMC) isolation, Rnase protection assay (RPA), PCR for HIV RNA and proviral DNA as well as quantitative image analysis (QIA). MMCs for flow cytometry will be stained for CD45, CD4, CD8, HLA-DR, CD45RO, CXCR4, CCR5, LFA-1, and ICAM-1.

Flow cytometry is performed on MMCs isolated from the mucosa and on PBMCs isolated from the blood to determine the composition of the lymphocyte populations in these compartments at baseline. The activation state and memory state of the lymphocytes will be noted, as will the expression of chemokine receptors and adhesion markers. These baseline parameters will be analyzed again after treatment with thalidomide.

β-chemokines and cytokines are quantified using the Multi-probe RPA (Riboquant, Pharmingen). This sensitive and specific assay allows quantification of chemokine and cytokine mRNA from extracted RNA from frozen biopsies. The quantity of the individual mRNA species will be compared with GADPH as a housekeeping gene. If chemokines and cytokines cannot be quantified with the RPA, they can be measured in supernatants of stimulated cultured MMCs by ELISA.

QIA utilizes immunohistochemical staining of paraffin-embedded tissue to accurately determine the number and anatomical position of lymphocytes bearing CD4, CD8, CD38, HLA-DR, CD45RO CXCR4 and CCR5 in a biopsy. In addition, it permits determination of which cells in a biopsy are producing cytokines and β-chemokines and the tissue concentrations of these factors. In HIV seropositive biopsies, the tissue levels of p24 can also be determined. A computerized image analyzer and specialized software are available to assess the total tissue area for stained cells, chemokines, cytokines, and p24 levels.

Results will confirm that the mucosal response to HIV is characterized by increased tissue chemokine and cytokine levels. This will, in turn result in a mild increase in the mucosal inflammatory infiltrate. By investigating chemokines and cytokines, “soluble” inflammation that would be overlooked by simply examining a cellular index will be confirmed.

Treatment with thalidomide, a potent immunomodulator will decrease mucosal inflammation in patients infected with HIV. This will likely manifest as a decrease in the ability of HIV to replicate in this, the largest lymphoid organ. Decreased replication in the mucosa, and perhaps systemically due to thalidomide will prevent the emergence of antiviral resistant HIV mutants and improve the long-term efficacy of HAART. As an adjunct to HAART therapy, thalidomide can further improve the life expectancy of the millions of patients worldwide infected with this virus.

Subjects with plasma undetectable HIV, having undergone baseline biopsies are treated with oral thalidomide therapy at a dose of 200 grams per day for 16 weeks. After 4 and 16 weeks of therapy, the subjects will undergo repeat endoscopic biopsies and repeat phlebotomy to obtain PBMCs and the same biomolecular experiments as described above are performed. In addition, one biopsy at each time point will be utilized for analysis of the patients mucosal viral load and plasma obtained by phlebotomy at each time point will be analyzed by the Roche ultrasensitive assay (detection level=40 copies of HIV RNA). RT- and DNA-PCR are performed to analyze changes between baseline and post-therapy mucosal and plasma viral load. For statistical evaluation, the solitary index of efficacy will be a decrease in mucosal HIV viral load without a concomitant increase in plasma viral load.

In addition, the effect of thalidomide therapy on the in vitro susceptibility to M-tropic and T-tropic HIV of MMCs and PBMCs isolated from the Crohn's disease patients with colitis before and after treatment with thalidomide will be examined. This study allows an evaluation of the effect of thalidomide on the ability of HIV to infect lymphocytes obtained from another inflammatory mucosal disease in vitro. This data will allow better correlates of the flow cytometric findings related to chemokine expression and levels of activation with changes in mucosal viral load. For infectivity assays, MMCs and PBMCs obtained at the baseline and 4 and 16 weeks of thalidomide therapy are cultured with known titers of M-tropic HIVsx and T-tropic HIVNL4-3. Supernatants are collected for ELISA to quantify p24 at time 0 hrs, 36 hrs, 3 days, and 7 days. Paired T tests to analyze changes in susceptibility of MMCs from IBD patients before and after therapy will be performed.

The treatment of HIV patients with thalidomide will alter the inflammatory environment of the gastrointestinal mucosa, resulting in a decrease in soluble inflammatory mediators (i.e., pro-inflammatory cytokines, chemokines), which in turn, will decrease recruitment of immune cells that HIV can infect. Reduction of inflammation by thalidomide therapy will impact p24 production in PBMCs and MMCs in the in vitro infection experiment. It is anticipated that the main benefit of thalidomide will be via decreased recruitment of additional co-receptor-bearing, CD4+ T cells as viral targets due to attenuated soluble inflammatory mediators in the mucosa, but it is possible that decreased expression of adhesion molecules may also play a role.

5-ASA Compounds or Products

Mesalamine (e.g., Asacol) is a mucosal anti-inflammatory medication taken orally or by enema, foam or suppository that is available for topical therapy. It will be a beneficial component of HIV therapy by decreasing HIV replication in gastrointestinal mucosa. Efforts to minimize mucosal inflammation with Mesalamine may be therapeutically beneficial by decreasing the concentration of pro-inflammatory cytokines and chemokines in the mucosal compartment and therefore recruitment and activation of additional cellular targets for HIV. By decreasing the concentration and activity of replicating HIV, it may also slow the development of HIV resistance to commonly used antiviral medications. Mesalamine can therefore be a powerful, locally active, adjunct in the treatment, inhibition of proliferation/spread within a subject having HIV, as well as inhibit or prevent HIV infection of a subject at risk of infection, and inhibit or prevent transmission of an HIV infected subject to another subject (uninfected or infected).

Subjects with known, moderately active infections based on clinical, endoscopic, and histologic examination but have not been receiving Mesalamine or other 5-ASA medications for 4 weeks and have not been receiving immunomodulatory medications or steroids (oral or topical) for 3 months will be studied. Non-colitic control patients will be age-matched to the colitis patients and be free of symptoms referable to their colon, and without endoscopic evidence of inflammation. Colitis subjects will be seen for 2 baseline endoscopic evaluations performed one-week prior and the day of initiation of therapy. Non-colitic control patients will also undergo two endoscopy examinations separated by one week. The soluble and cellular inflammatory state of mucosal samples from the ulcerative colitis and non-colitic controls will be compared.

Fifteen endoscopic biopsies (3.3 mm OD) from a standardized level (30 cm) will be obtained from each patient. Of the biopsies, 12 will be used for MMC isolation, 1 for RPA, and 2 for QIA. MMCs for flow cytometry will be stained for CD45, CD4, CD8, CD38, HLA-DR, CD45RO, CCR5, and CXCR4.

β-chemokines and cytokines will be quantified in supernatants obtained after culture of MMCs and PBMCs in IL-2 containing medium by ELISA. One million cells in 1 ml medium in 6 well plates will be used. After 18 hours of culture, 200 ml medium will be taken for measurement of RANTES, MIP-1a, MIP-1b, IL-1b, IL-12, TNF-α, and IFN-γ. If needed, nucleic acid will be extracted from a liquid nitrogen frozen biopsy to determine the level of mRNA for these cytokine and chemokine species using the Multi-probe RPA (Riboquant, Pharmingen). The quantity of the individual mRNA species will be compared with GADPH as a housekeeping gene.

QIA utilizes immunohistochemical staining of fresh frozen or paraffin-embedded tissue to accurately determine the number and anatomical position of lymphocytes bearing CD4, CD8, CD38, HLA-DR, CD45RO, and CCR5 in a biopsy. In addition, it permits determination of which cells in a biopsy are producing cytokines and β-chemokines, the tissue concentrations of these factors, and by inference, the effect of these ligands on chemokine receptor expression of neighboring cells. A computerized image analyzer and specialized software are available to assess the total tissue area for stained cells, chemokines, and cytokines (expressed as % area).

Moderately severe ulcerative colitis is characterized by elevated levels of chemokines and cytokines “soluble inflammation” as well as the known increased cellular inflammation. The mucosa in ulcerative colitis will be characterized by soluble and cellular inflammation that is significantly greater than in non-colitic controls. The ability of Mesalamine to reduce soluble and cellular inflammatory mediators is examined.

Treatment with Mesalamine will result in cytokine and chemokine levels being decreased, minimizing recruitment of CCR5-bearing CD4+ cells and lateral spread of HIV among mucosal lymphocytes; Mesalamine-induced decreases in β-chemokine secretion may reduce the amount of ligands able to block the viral co-receptors, favoring the spread of HIV; Mesalamine will preferentially inhibit pro-inflammatory cytokines resulting in suppression of T cell recruitment while not significantly impairing chemokine receptor blockade. This appears to be the most beneficial outcome when treating HIV-infected patients. The possible outcomes are examined by treating patients with moderately active inflammatory bowel disease (IBD) with Mesalamine for 16 weeks and examining the changes in mucosal lymphocyte subsets, expression of chemokine receptors, and mucosal cytokine and β-chemokine levels.

Subjects with moderately active ulcerative colitis, having undergone two baseline biopsies are treated with Mesalamine at a dose of 4.8 grams per day for 16 weeks. After receiving 4 weeks of therapy and again after completion of 16 weeks of therapy, they will undergo repeat endoscopic biopsies and the experiments are repeated on the post-therapy specimens. These studies will include performance of flow cytometry to quantify lymphocyte subsets and chemokine receptor expression, RPA to quantitate β-chemokine mRNA and QIA for in situ analysis. Patients will continue to be managed on Mesalamine or as clinically indicated by their physicians.

The treatment of IBD patients with Mesalamine will alter the inflammatory environment of the gastrointestinal mucosa, resulting in a decrease in soluble and cellular inflammatory mediators.

HIV infection of an activated CD4+, chemokine receptor bearing cell results in heightened HIV replication. The inflammatory environment that characterizes the gastrointestinal mucosa likely induces vigorous HIV replication. Therapy with Mesalamine, by reducing inflammation, will down-regulate the ability of HIV to replicate in the inflamed gastrointestinal mucosa.

To examine the effect of Mesalamine therapy on HIV replication in mucosal mononuclear cells, the susceptibility of MMCs and PBMCs, from untreated ulcerative colitis patients, to M-tropic and T-tropic HIV infection is examined in culture with and without the presence of graded doses of Mesalamine. Similar infectivity experiments will be conducted on the patient's cells after they have received 4 and 16 weeks of Mesalamine therapy.

For infectivity assays, MMCs and PBMCs obtained at the baseline time-points will be cultured with known titers of M-tropic HIVSX and T-tropic HIVNL4-3 in the presence and absence of graded, but physiologic concentrations of Mesalamine, based on known mucosal tissue concentrations. HIV replication will be compared in each of these samples by measurement of production of the HIV protein p24 in the culture supernatants. MMCs and PBMCs from subjects after 4 and 16 weeks of therapy will be cultured with M- and T-tropic HIV without added Mesalamine. The p24 results gathered from these samples will be compared with the baseline samples. In each of these experiments, supernatants will be collected for ELISA to quantify p24 at time 0 hrs, 36 hrs, 3 days, and 7 days. Paired T tests will be used to analyze changes in susceptibility of these cells to HIV.

MMCs will be more susceptible to HIV than are PBMCs due to the activated CCR5+ phenotype as we already shown. Heightened mucosal inflammation in IBD will further facilitate HIV infection of MMCs. Reduction of inflammation by Mesalamine therapy will impact p24 production more in MMCs than in PBMCs.

The mucosal environment in HIV is inflammatory as evidenced by elevated concentrations of pro-inflammatory cytokines and chemokines. Mesalamine will significantly reduce these soluble mediators and in doing so, reduce the migration into and activation of further cellular targets for HIV infection into the mucosa.

### EXAMPLES

Among the multiple co-receptors that HIV-1 is able to utilize, CCR5 and CXCR4 play a major role, with CCR5-tropic viruses predominating during initial infection, CXCR4-tropic viruses becoming more prevalent with advanced disease and heterozygosity of CCR5 contributing to longer survival. Differential expression of CCR5 on mucosal CD4+ T lymphocytes could contribute to preferential transmission of M-tropic viruses. In order to compare co-receptor expression on mucosal versus circulating lymphocytes, mucosal mononuclear cells (MMC) were isolated from rectosigmoid endoscopic biopsies and obtained unstimulated phlebotomy samples from HIV-1 seronegative healthy individuals. We quantified co-receptor expression on CD4+ cells by flow cytometry.

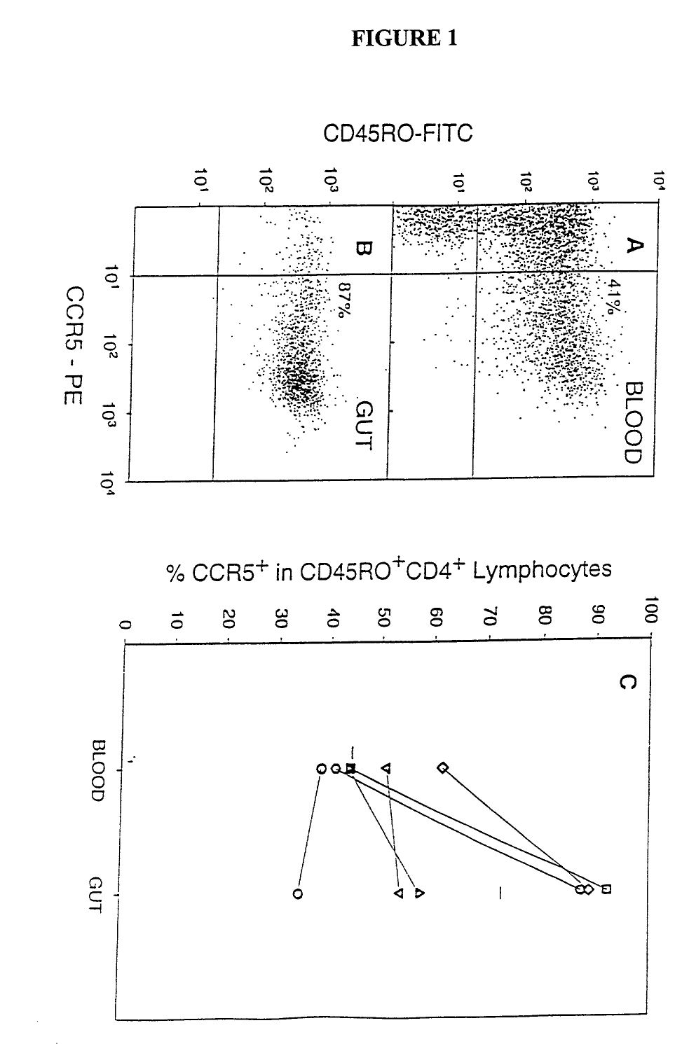

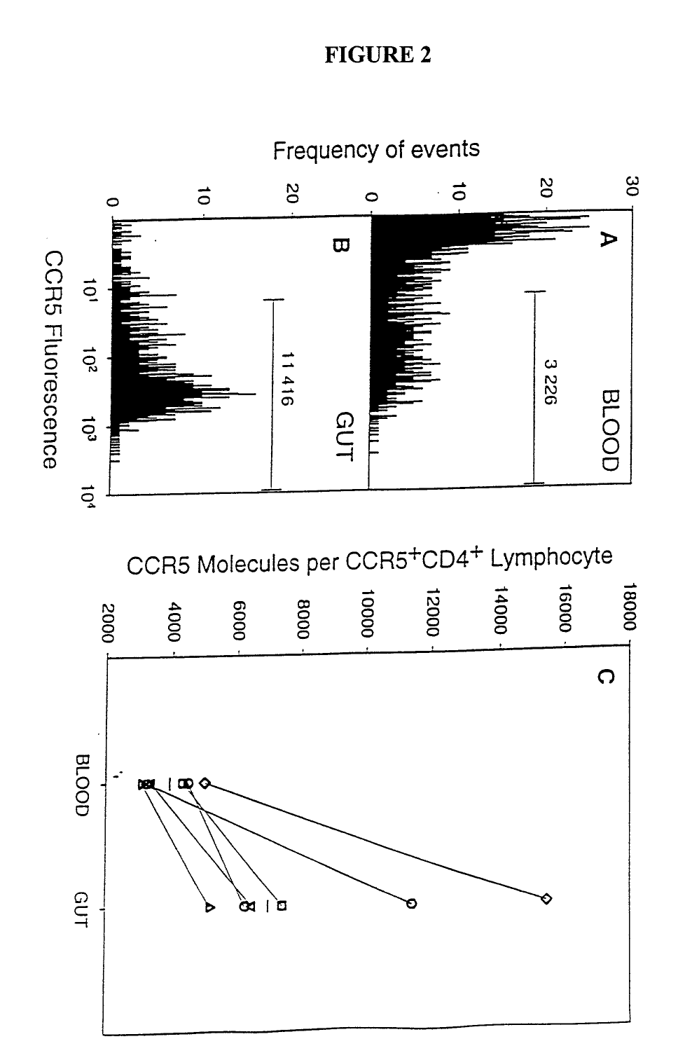

In agreement with published studies, a median of 23% (interquartile [i.q.] range 18-30%) of all CD4+ lymphocytes in blood expressed CCR5. As shown in FIG. 1, a median of 71% (i.q. range 50-87%) of the CD4+ lymphocytes in the gut expressed CCR5, a 2.8-fold greater percentage than in the blood (P=0.03). Mucosal CD4+ lymphocytes also expressed significantly more CCR5 receptors per cell than did their CCR5-expressing CD4+ lymphocyte blood counterparts, further extending the compartmental difference. As shown in FIG. 2, the median CCR5 receptor number per CD4+ mucosal lymphocyte was 6,946 molecules (i.q. range 6,306-10,416) compared to approximately 3,841 (i.q. range 3,259-4,441) CCR5 receptors per CD4+ blood lymphocyte, a 2.2-fold increase (P=0.03). Taken together, this translates into a 6.2-fold increase in total expressed CCR5 receptors potentially available for viral access on CD4+ lymphocytes in the gut compared to the blood. These findings suggest that mucosal CD4+ lymphocytes may be much more vulnerable to infection by M-tropic HIV-1 than their blood counterparts.

Nearly all (97%) of the CCR5 expression on CD4+ lymphocytes in both the blood and gut was on cells of the memory CD45RO+ phenotype. In agreement with published studies, we found an increased proportion of CD45RO+ memory cells among gut CD4+ lymphocytes (median 95%; i.q. range 90-97%) compared to blood (median 46%; i.q. range 38-53%).

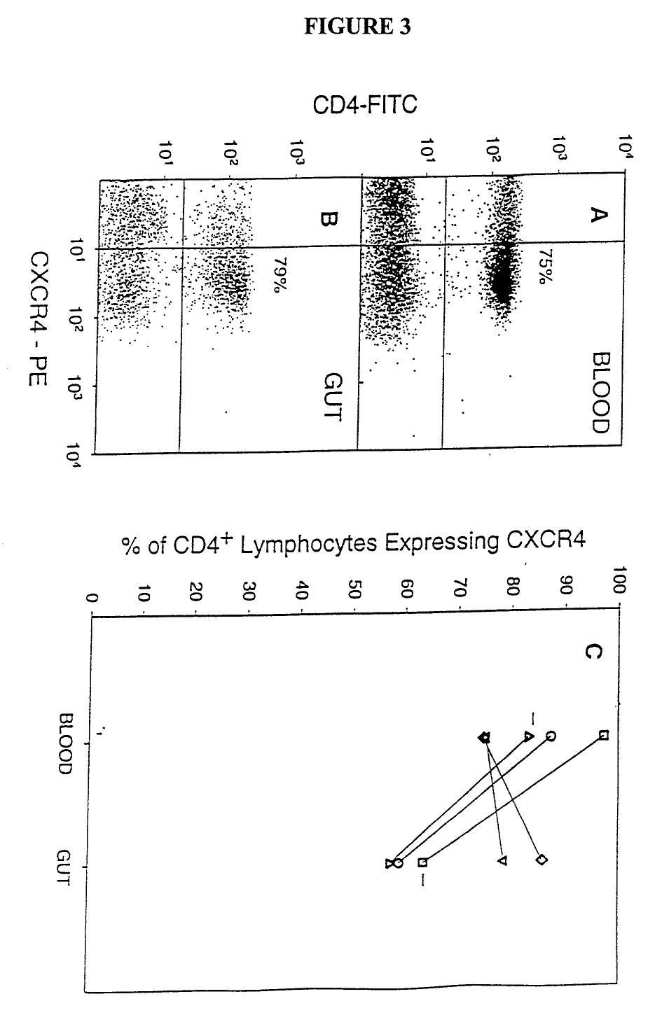

It has been previously reported that most peripheral T cell CXCR4 is expressed on naïve CD45R0− but not on memory CD4+RO+T cells. Therefore, as initially expected the high levels of CCR5 expression and the predominantly CD45RO+ phenotype of the MMC might provide an anatomic and cellular mechanism that might explain preferential transmission of M-tropic viruses at least during anal-receptive intercourse. However, also found were high levels of CXCR4 expressing cells both in blood (median 83%; i.q. range 75-87%) and gut (median 64%; i.q. range 59-79%), as shown in FIG. 3. There was no significant difference between the two compartments either in percentage (P=0.03) or in relative fluorescence intensity of staining with anti-CXCR4 antibody. In three donors, a sufficient cell yield was obtained to analyze CXCR4 expression on memory CD4+ lymphocytes and confirmed that a substantial fraction of both blood memory cells (median 69%; i.q. range 57-73%) and gut memory cells (median 62%; i.q. range 57-65%) expressed detectable levels of CXCR4. By comparing the relative fluorescence intensity of expression of CXCR4 on blood memory (CD45RO+) and naïve (CD45RO−) CD4+ cells, estimates indicate that levels of expression of CXCR4 on memory CD4+ cells are only slightly lower than the levels on naïve CD4+ cells. Our results indicate that memory CD45RO+ cells can express not only CCR5 but also CXCR4 at levels most likely adequate to facilitate infection.

Our finding of high levels of expression of co-receptors for HIV-1 infection in humans contrasts with a study in macaques in which low numbers of CXCR4-expressing cells were identified on rectal and colonic tissue sections. Even more sparse in the macaque study were CCR5-bearing T cells and macrophages. The differences may reflect a species difference or differences in acquisition of the tissues. Our findings are based on isolated viable cells using a method found to preserves cell surface expression of CCR5 and CXCR4.

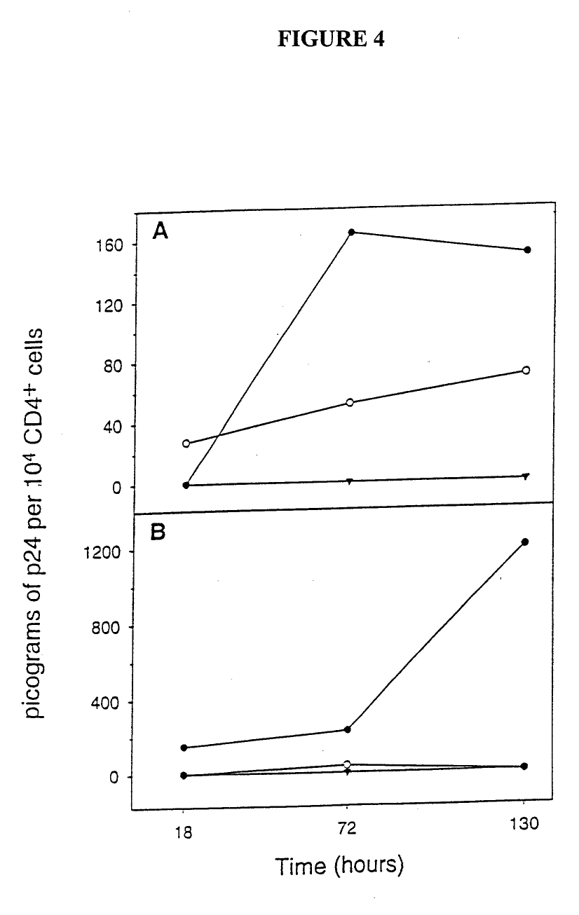

In order to evaluate the susceptibility of mucosal mononuclear cells to HIV-1 infection, we subjected isolated MMC from endoscopic biopsies and isolated PBMCs from healthy HIV-seronegative volunteers to in vitro HIV infection. Infection of mucosal cells with laboratory strains of HIV (M-tropic HIVSX or T-tropic HIVNL4-3) was performed in the presence of 20 IU of interleukin-2 (IL-2), as data had shown that IL-2 was required to maintain viability of mucosal cell populations. As a control, since IL-2 is known to upregulate CCR5 and could enhance viral replication, PBMCs were also infected from the same patient both with and without IL-2. Infection was quantified at 18 hours, 72 hours, and 130 hours by p24 production in the supernatant and expressed in terms of pg of p24 produced per 104 CD4+ lymphocytes. Mucosal mononuclear cells were able to support vigorous viral replication in culture compared to PBMC with or without IL-2 as shown in FIG. 4 in a representative experiment (one of two). PBMC infected in the absence of IL-2 could not support HIV replication by either HIVSX or HIVNL4-3. When compared with similarly cultured PBMCs in the presence of IL-2, mucosal cells were markedly more susceptible than PBMC to M-tropic and T-tropic HIV. For example, at 72 hours, supernatant p24 levels of the M-tropic HIVSX in the MMC culture was 164 pg/ml per 104 CD4+ lymphocytes compared with 51 pg/ml in the PBMC culture. For T-tropic HIVNL4-3, viral growth accelerated over time and at 130 hours, supernatant p24 levels in the MMC cultures was 1194 pg/ml per 104 CD4+ lymphocytes compared to undetectable levels in cultures of PBMC. These data indicate that the enhanced vulnerability to infection suggested by the mucosal CD4+ lymphocyte co-receptor phenotype renders them functionally injectable in vitro by both M and T-tropic strains of HIV-1.

Three points of interest are raised by these data. First, tissue-based immune cells can be easily and safely obtained and isolated from the mucosal lining of the gut, a renewable tissue source. As studies of HIV-1 pathogenesis increasingly focus on tissue compartments for both persistence and transmission studies, techniques to easily and safely access lymphoid tissue are essential. Lymph node and tonsillar resection/aspirations have been the most commonly reported methods. Studies using these approaches have already provided illuminating, concept-changing findings including evidence that HIV-1 activity persists in lymphoid tissue when plasma levels are stable or undetectable. These tissue sources reveal the biologic events that occur in secondary, organized lymphoid structures during HIV-1 infection, but require invasive surgical support for tissue acquisition. In contrast, the gut mucosal lymphoid tissue is abundant, easily accessible, quickly healing, self-replenishing, and directly visible. Endoscopic biopsies are safe, quick, painless, and provide access to the lymphoid compartment with 100% of samples containing lymphocytes. The biopsies maintain architectural orientation and can be examined histologically, or can be dissociated for flow cytometric and tissue culture evaluation as described here.

A second point of interest raised by these studies is that CCR5 expression is markedly increased on human mucosal CD4+ lymphocytes, both as a percentage of total CD4+ lymphocytes and on a per cell basis compared to peripheral blood cells. These mucosal T cells support higher levels of viral replication than CD4+ lymphocytes from blood (FIG. 4). As CCR5 is the co-receptor most associated with HIV-1 in early infection, and the gastrointestinal tract is one of the most common sites of transmission as well as being the body's major lymphoid organ, the amount of detected CCR5 expression carries important implications for transmission, primary infection, ongoing local spread and treatment. Projections of T cell infectability by M-tropic HIV-1, based on CCR5 expression in blood, would lead to a dramatically different mathematical modeling of disease progression. When CD4 was not a limiting factor, a minimum of 700-2000 CCR5 receptors per cell was adequate for maximal susceptibility to infection. By our calculations, the number of CCR5 receptors on blood T cells (median of approximately 3000 per cell) would be within this range of in vitro infectability. The mucosal levels (median of approximately 7000 receptors per cell) far exceed this minimal range. Factors including b-chemokine production levels and presence of cellular activation factors in the MMC could also impact the capacity of these cells to support replication. Cytokines including TNF-a and IL-2 are found at high levels in gut mucosa and may also contribute to enhanced viral replication at this site.

A third point of interest is the enhanced sensitivity of mucosal lymphocytes to infection with both CCR5- and CXCR4-tropic strains of HIV-1 compared with PBMC. The extraordinarily high levels of HIVNL4-3 production in MMC cultures in the presence of IL-2 but without further stimulation of the cells suggests that mucosal T cells provide a rich milieu for replication of variants of HIV-1 that use either of the major co-receptors for entry. Why CCR5-using variants are preferentially transmitted despite the abundance of CD4+ lymphocytes at the site of mucosal transmission of HIV-1 and the susceptibility of these lymphocytes to infection by both types of virus remains an important question to answer. Our findings raise the question of whether HIV-1 variants using CXCR4 as a co-receptor are initially transmitted but are cleared, perhaps through an immunologic mechanism. Alternatively, it is possible that CXCR4-using variants could be retained at the site of transmission while CCR5 using viruses spread to PBMC.

Our results show that tissue biopsies of gut mucosa can be used to obtain quantitative information on immunologic and virologic determinants that may influence HIV-1 transmission and pathogenesis. The potential vulnerability to primary and persistent HIV-1 infection of the gastrointestinal mucosa, a sexually exposed and easily traumatized lining, is dramatic. This vulnerability includes the predominance of activated memory CD4+ lymphocytes at this mucosal site. Our results also show that both CCR5 and CXCR4 are highly expressed on mucosal CD4+ lymphocytes from the gastrointestinal tract in healthy, HIV-1-seronegative individuals, and these mucosal cells are highly susceptible to infection in vitro, much more so than cells from the blood.

Patient population: Six healthy individuals, three men and three women (mean age 45, range 24-68), were recruited for the study of lymphocyte phenotypes. Two additional men were recruited for the viral culture experiments (see below). Informed consent was obtained prior to undergoing elective endoscopy for a history of blood in stool or routine polyp screening. No subjects had diarrhea symptoms or history of intestinal inflammatory or infectious disorders. Hematoxylin and eosin stained biopsies taken in the same area as study biopsies revealed no pathology and were all normal appearing when reviewed in a blinded fashion by a gastrointestinal surgical pathologist. The study was approved by the UCLA Human Subjects Protection Committee. A site of 30 cm in the rectosigmoid colon was used routinely for all sampling to avoid potentially confounding inflammation resulting from traumatic or infectious proctitis. Mucosal mononuclear cells (MMC) were isolated from four endoscopic biopsies from each donor. Biopsies were collected using 3.3 OD forceps into 15 ml of tissue culture medium (RPMI 1640, Irvine Scientific). The biopsies were maintained at room temperature on a rotating platform until isolation (roughly 20-60 minutes) then removed to a 10×35 mm petridish containing phosphate buffered saline (PBS) with 1 mM EDTA and 50 mM 2-mercaptoethanol and the samples teased apart using 18G needles. The disrupted tissue was incubated at 37□ C. for 20 minutes in a shaking water bath. Following centrifugation, the tissue samples were digested with a mixture of collagenase and dispase (Boehringer Mannheim #269638; 0.1 mg/mL in RPMI) for 1 hour at 37□ C. Further disruption was achieved by sample passage through syringes with a series of decreasing needle gauges. Debris was removed using a 70 micron cell strainer (Falcon #2350). Resulting cells were resuspended in RPMI containing 10% fetal calf serum. Mononuclear cells, which included primarily epithelial cells and leukocytes, were counted visually using a hemocytometer and the proportion of mononuclear cells that were leukocytes was estimated. About 20% of the mononuclear cells were leukocytes from a yield of mean 1.3×106±1.1×106 S.D. (n=6) per four biopsies. Viability, determined by the exclusion of trypan blue, was >90%. Blood from the donors was collected in EDTA and was stained using the whole blood staining method.

Monoclonal antibodies purchased included CD4-fluorescein isothiocyanate and CD45-peridin chlorophyl protein (BDIS), CD8-allophycocyanin (Caltag), and anti-CXCR4-R-phycoerythrin (PE; Pharmingen). Anti-CCR5 was provided by Dr. Walter Newman of Leukosite, (Cambridge, Mass.) and was prepared as a 1:1 conjugate with PE by Drs. Kenneth Davis and Noel Warner of BDIS. Analysis was carried out on a FACSCaliburÒ (BDIS) with analysis using Cell QuestÒ software. Initial gating on the isolated MMCs was performed using side scatter and CD45 fluorescence followed by forward and side scatter gating. A well-defined and separate population of mucosal leukocytes was identified as CD45bright, and represented about 10-50% of the initial mononuclear sample population. Of these, 20-40% were CD4+ lymphocytes and 26-41% were CD8+ T cells.

To estimate the number of CCR5 molecules per CD4+ lymphocyte, the observed CCR5 relative fluorescence intensity (RFI) was multiplied by a calibration factor, specifically 44, determined for our FACSCalibur. This calibration factor is the number of molecules of PE detected per RFI channel number. For mAb prepared as 1:1 conjugates with PE, the RFI channel number can be multiplied by the calibration factor to estimate the number of mAb bound per cell. This calculation was not performed for CXCR4 because it was not available as a 1:1 conjugate.

To ensure that the collagenase/dispase isolation process used on the gut biopsies did not degrade nor strip surface antigens, peripheral blood mononuclear cells (PBMC) were isolated by Ficoll-Hypaque separation and then either stained and analyzed directly by flow cytometry for percent CD45, CD4, CD8, CCR5 and CXCR4 expression or processed through the mucosal isolation procedure (collagenase/dispase treatment) and then stained and analyzed for the antigens. PBMC routinely isolated and those exposed to mucosal isolation enzymes showed no discernible differences in quadrant percentages for all antibodies studied. Thus, the observation of increased percentages and expression of CCR5 on cells from the gut compared to those from the blood does not result from the isolation process since CCR5 expression was not increased by treatment with collagenase/dispase.

MMCs from each healthy, HIV-seronegative volunteer were isolated from four endoscopic mucosal biopsies after mechanical disruption followed by 3-day culture in Iscove's DMEM medium supplemented with 10% human serum, containing 10 mg/ml gentamycin, penicillin, streptomycin and glutamine. Interleukin-2 (IL-2, Amgen) was added at 20 IU per mL. A total of 105 mucosal mononuclear cells and PBMCs were plated in a 96-well plate in 100 microliters of medium after a 3-hour infection with 50 mg of HIVSX or HIVNL4-3. Prior to plating, the cells were washed twice to remove free virus and adherent p24. Thirty microliters of supernatant was sampled at each time point 18 hours, 3 days (72 hours), and 5 days (130 hours) for p24 measurement by ELISA (Coulter). CD4+ percentages were determined by flow cytometry and were used to determine the number of CD4+ lymphocytes in the cultures. Co-receptor expression on the gut cells was not tested on these donors because cells yields were not sufficient.

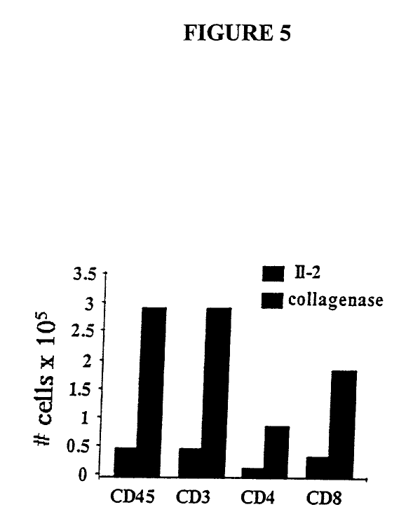

In an attempt to increase the yield of mucosal mononuclear cells for functional, infectivity and flow studies, an alternative isolation method was undertaken. Freshly collected endoscopic biopsies are minced directly into 10 ml of Iscoves Medium supplemented with 20 units/ml of IL-2 in a 100×300 mm petri-dish and cultured for 3 days in 5% CO2 at 37 degrees. Cells are harvested through a 70 μm cell strainer and the total mononuclear cell yield enumerated visually by hemocytometer. The yield of CD45+, CD3+, CD4+ and CD8+ cells was determined using TruCount beads and was compared to the yield from biopsies collected from the same individual isolated using the conventional collagenase/dispase protocol. There is a 6-fold increase in the yield of mucosal lymphocytes. (FIG. 5).

Quantitation of HIV-1 in Tissue.

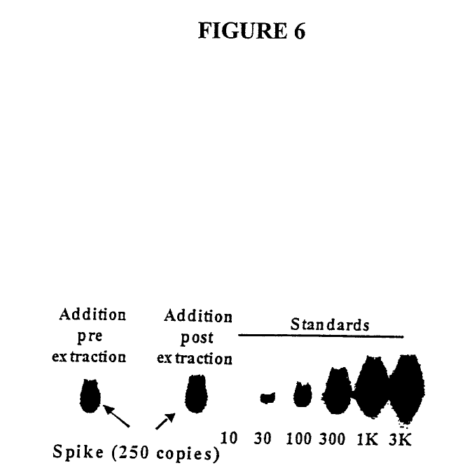

Extraction of RNA from rectosigmoid biopsies results in >95% recovery of tissue RNA. We have developed a quantitative RT PCR assay for tissue RNA and quantitative PCR for tissue DNA, adapted from that previously described by the Chen laboratory. Our initial results show that HIV-1 RNA can be quantitated by RT PCR to levels as low as 10 copies (FIG. 6).

Samples are immediately homogenized from the frozen state (using Powergen 125 tissue homogenizer), Trizol-extracted with separation of RNA and DNA containing phases. RNA is further extracted using an Rneasy column. Quality control studies have confirmed minimal RNA degradation (agarose gel electrophoresis) and no DNA contamination (PCR of RNA template). The number of HIV RNA copies is quantitated using an adaptation of the rTTH RNA PCR kit (Perkin-Elmer) with HIV LTR specific primers 667/AA55 designed to capture unspliced/multiply spliced HIV RNA. A linear standard curve is generated using a 127 bp sequence recognized by the 667/AA55 primer pairs. DNA is isolated by ethanol precipitation with at least 2 washes in 0.1M sodium citrate/10% ethanol buffer. For HIV DNA, the same primer pairs are used for PCR amplification (667/AA55). Linear standard curves have been generated using β-globin primers.

In an effort to standardize our approach and have resultant yields most closely reflect in vivo amounts of HIV RNA, known quantities of HIV LTR RNA were added to seronegative biopsies both pre and post nucleic acid isolation. A linear standard curve was generated using purified HIV LTR RNA diluted in 0.5 ug/ul of Hela-cell total RNA and RT-PCR was performed as previously described. The difference between the pre and post LTR-supplemented samples was quantified and found to be minimal (>95% recovery).

For quantitative assessment of recovery, known quantities of luciferase DNA, a bacterial sequence with no known human homology, to quantitate tissue DNA recovery (usually >75%); seronegative samples receive a known quantity of LTR HIV sequence to quantitate RNA recovery (>95%).

HIV-RNA is Reproducibly Detected in Rectosigmoid Biopsies from Subjects with Undetectable Plasma Viral Load.

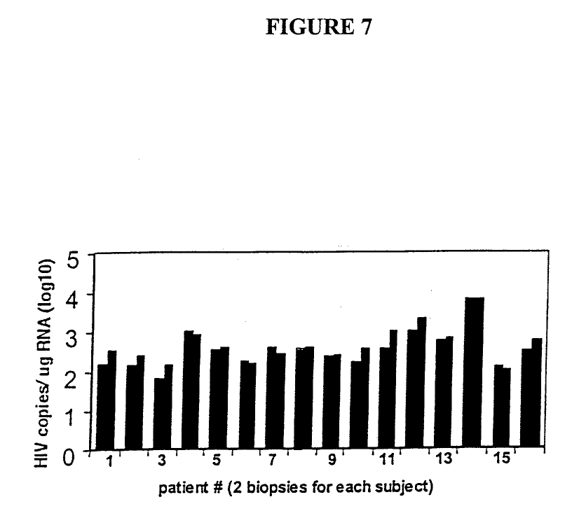

Efforts were made to demonstrate the replicability of results from one biopsy by comparison with others concurrently obtained at the same circumferential level (30 cm) from the same patient. Single biopsies (10 mg each) from subjects with undetectable plasma viral loads were frozen, RNA extracted and amplified using LTR-specific primers 667/AA55, as described above. Each biopsy yielded an average of 25 μg RNA of which usually 1/100 was used for quantitation. Results in FIG. 7 demonstrate the reproducibility of quantitated RNA viral burden using rectosigmoid biopsies in a sensitive assay. The data demonstrates the tissue HIV RNA viral load from 2 biopsies obtained during the baseline sigmoidoscopies for subjects with undetectable plasma viral loads. These individuals reported undetectable plasma viral load for >1 year. On average, there is <0.2 log 10 difference between samples within the same subject. These data show the reproducibility and minimal sample to sample variation in using biopsies to quantitate tissue viral load. Equally important is the demonstration of detectable levels of tissue HIV RNA (usually 102 to 103 per μg RNA) in subjects with undetectable plasma HIV RNA.

HIV-DNA is Reproducibly Detected in Rectosigmoid Biopsies from Subjects with Undetectable Plasma Viral Load.

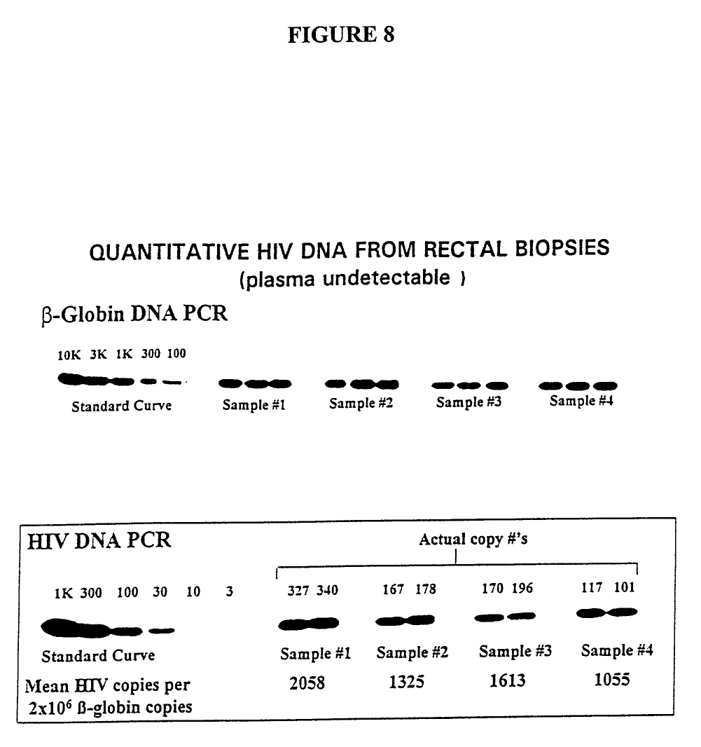

In a separate group of subjects with undetectable plasma viral load, HIV DNA was amplified using quantitative PCR with specific 667/AA55 primers for the LTR region and b-globin specific primers used for internal linear standard. Triplicates were assayed for the b-globin specific primers; duplicates of the HIV proviral DNA quantitation are shown in FIG. 8. Although lower limit of detection is 3 copies, 10 copies were used as our lower cut-off point. The figure shows both the actual copy number quantitated and the calculated number of copies based on a b-globin-dependent cell count. These results show our technique can detect copies of proviral DNA as low as 10 in subjects with undetectable plasma viral load.

CCR5 Co-Receptor Expression on Mucosal CD4 T Cells.

Isolation of mucosal mononuclear cells does not alter phenotypic expression of CD4, CD8, CCR5, and CXCR4.

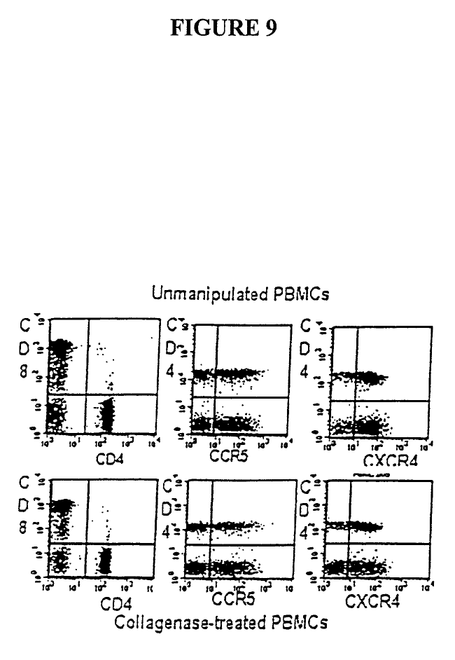

Samples were isolated from healthy, seronegative controls' blood (peripheral blood mononuclear cells: PBMC) and intestinal mucosa (mucosal mononuclear cells: MMC) to establish baseline CCR5 and CXCR4 expression in both compartments. FIG. 9 demonstrates that our isolation procedure neither strips relevant receptors (CD4, CD8, CCR5, CXCR4) nor alters their surface expression.

Mucosal expression of CCR5 on CD4 T cells is greatly increased compared with PBMC. Isolated mucosal mononuclear cells and peripheral blood mononuclear cells were obtained from healthy, seronegative control subjects and evaluated to determine the relative percentages of CD4 T lymphocytes in each compartment expressing CCR5 receptors. The 2D7 CCR5 antibody is conjugated in a 1:1 ratio with phycoerythrin; the flow cytometry instruments used are calibrated to detect 44 phycoerythrin molecules per RFI channel (based on a standardized CD4 expression and number of antibodies bound per cell). Consequently, the number of anti-CCR5 antibodies bound per cell can be translated to number of receptors per cell, assuming monovalent binding of antibody to receptor.

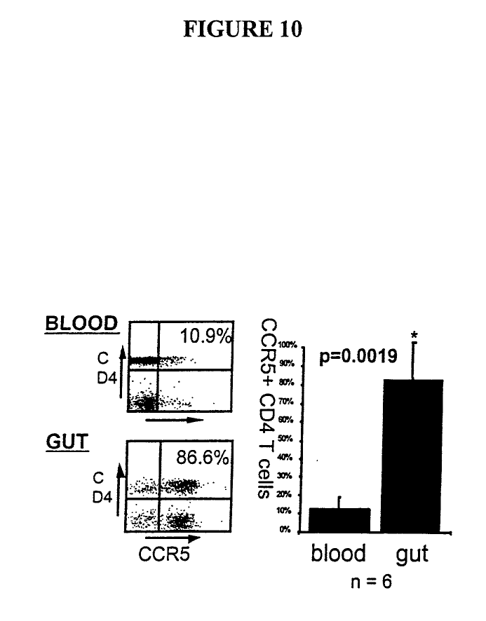

The percentages of CCR5-expressing CD4+ T cells is significantly increased in the gut (87%) compared to the blood (11%) (p=0.0019) (FIG. 10).

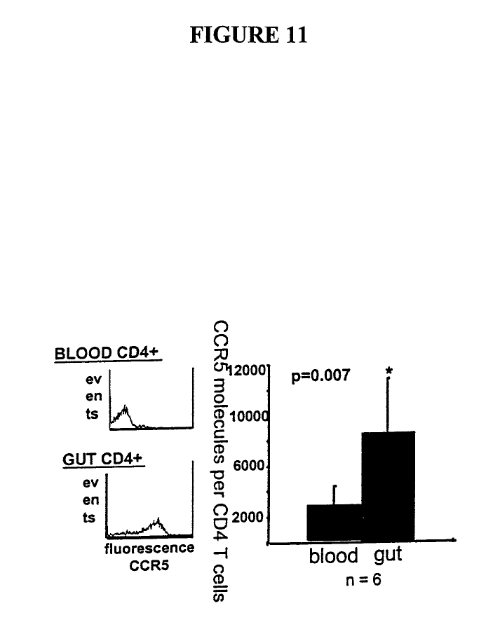

Further enhancing the vulnerability to HIV infection, FIG. 11 shows mucosal CD4 T cells also express significantly more receptors per cell (mean of 8500) compared to blood CD4 T cells (mean 2700)(p=0.007).

CCR5 expression on mucosal CD4 T cells is nearly exclusively on the memory subset. Blood and mucosal samples from the same seronegative, healthy controls were counter stained with CD45RO antibody as an indicator of the memory subset to determine the relative distribution of CCR5 staining. After gating on CD4+ fluorescence, 91% of CD4+ CD45RO+ mucosal cells express CCR5 compared to 24% of a matched group in the blood (p=0.017).

CCR5 Expression on Mucosal CD4 T Cells Remains Increased Compared to PBMC in HIV-Infected and Inflammatory Samples.

Having ascertained preliminary baselines of CCR5 expression in mucosal and blood CD4 T cells in healthy, seronegative subjects, the expression on CD4 T cells in the setting of chronic HIV infection was evaluated to test the hypothesis that CCR5 expression would remain increased in the mucosa compared to blood, favoring HIV replication. Inflammatory controls were included to discern changes not directly related to HIV-infection. Inflammatory controls were well-characterized subjects with inflammatory bowel disease (IBD), specifically, ulcerative colitis. Subjects were clinically in remission (maintained but controlled mucosal inflammation) on 5-ASA anti-inflammatory agents only, no steroids or immunosuppressive medications were used. HIV-infected individuals had peripheral CD4 counts between 200-700 cells/mm3 with a range of plasma viral loads (undetectable by ultrasensitive assay: n=2; plasma viral load between 200-2000 copies/ml: n=2; plasma viral load between 20,000-40,000 copies/ml: n=4).

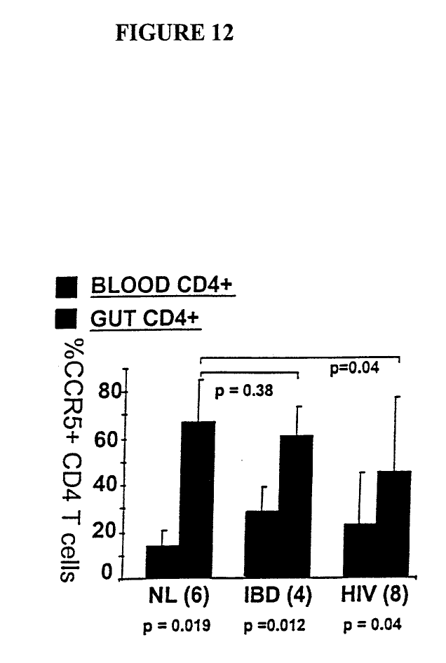

The differential expression of CCR5 between mucosal and blood CD4 T cells observed in seronegative normal controls was maintained in inflammatory controls (p=0.012) and HIV-infected subjects (p=0.04)(FIG. 12). In agreement with our hypothesis, there is a trend toward significance identifying a decrease in mucosal CCR5 expression on CD4 T cells in HIV compared with normal controls as shown in this figure.

The CD4:CD8 Ratio of CCR5-Expressing T Cells Decreases in HIV and IBD in Both Blood and Gut.

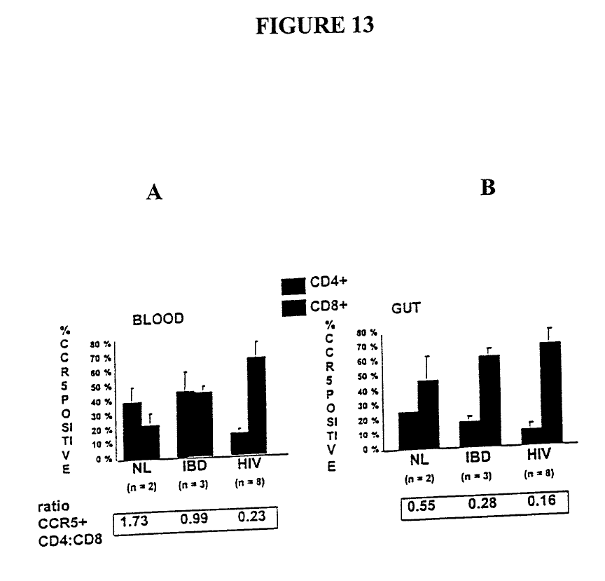

CCR5 receptor on CD4 T cells are also expressed on CD8+ T cells. To further evaluate if there was a true decrease in CCR5 expression on mucosal CD4 T cells, evaluation of the relative distribution of CCR5 receptors between CD4 and CD8 T lymphocytes in both compartments in the three clinical conditions. FIG. 13 demonstrates the dramatic downward shifts in the CD4:CD8 ratios of CCR5 expressing cells, decreasing in blood and gut samples by roughly 50% in IBD and 70-90% in HIV. This may represent a protective down regulation of CCR5 to inhibit HIV spread. Given the similar trend in the inflammatory controls, the primary stimulus for decreased surface expression likely relates to the inflammatory milieu. Samples are being processed to confirm that b-chemokine levels are elevated in both conditions, but even more so in HIV than in IBD samples. Supporting our hypothesis, these findings would suggest an extremely active inflammatory mucosal state in HIV (as defined by chemokine activity) despite the histological reports of relative lymphopenia.

Quantitation of β-Chemokine Tissue Concentrations Using QIA.

To further assess our hypothesis that HIV infection provokes a significant mucosal inflammatory response, pilot studies were conducted with Dr. Jan Andersson at the Karolinska Institute to quantitate chemokine concentrations in tissue. Endoscopic biopsies that were quickly oriented on foil and snap frozen were sent to Sweden on dry ice for cryosectioning (7 μm) and quantitative immunohistochemical staining with antibodies to identify RANTES, MIP-1a and MIP-1b. Quantitative image analysis (QIA) was also performed using CD4, CD8, and CCR5 antibodies. Samples from a healthy, seronegative control and an HIV-infected individual with a detectable plasma viral load were studied. Results are expressed as the percentage of total tissue area measure that was positively stained with the peroxidase-labeled antibody. The findings in the healthy control for RANTES, MIP-1a and MIP-1b were 2.04%, 1.39% and 1.65% respectively. In the HIV-infected subject's sample, the respective value increased to 15.1%, 6.2% and 12.1%. These chemokines function to recruit additional inflammatory cells into the already inflammed mucosa. This dramatic increase that HIV infection is associated with mucosal inflammation is provided in Table 1.

TABLE 1

| Chemokine | Normal Control HIV-infected | |
|---|---|---|
| RANTES | 2.04% | 15.1% |
| MIP-1a | 1.39% | 6.2% |
| MIP-1b | 1.65% | 12.1% |

Mucosal Mononuclear Cells (MMC) are Significantly More Infectible in vitro than PBMC.

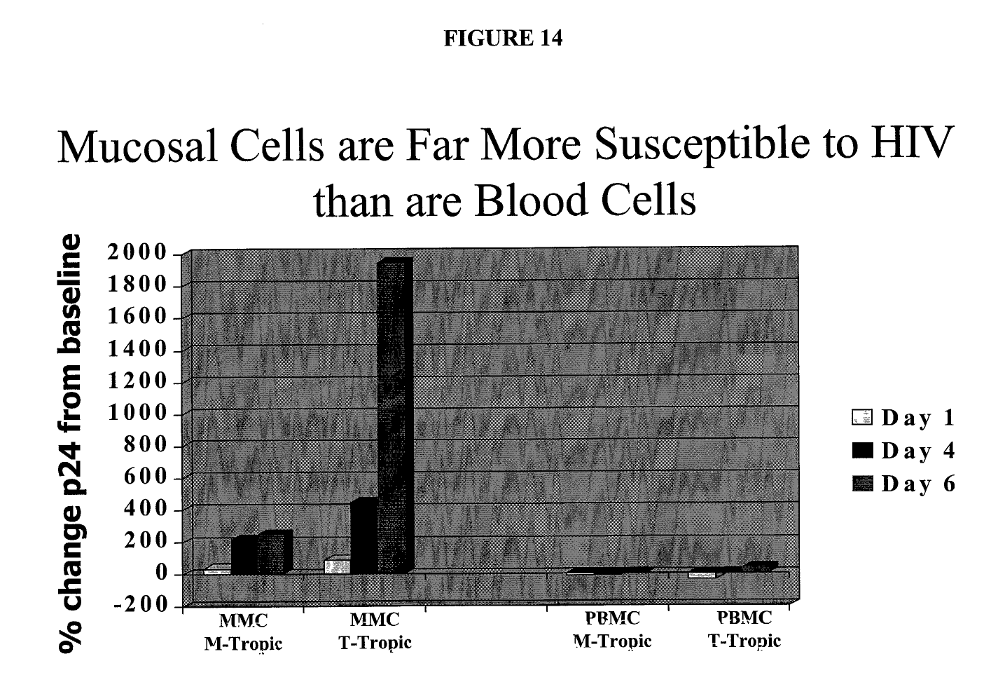

Initial observations demonstrated increased vulnerability to HIV infection of mucosal cells due, in part, to increased co-receptor expression. This hypothesis was tested in vitro using isolated MMC and PBMC from the same individuals and incubated with M-tropic HIVSX for 2 hours, washed and cultured for 3-10 days. Aliquots of supernatant were collected at the demonstrated times and assayed for p24 production as evidence of infection. The p24 production at the first time point (3 days) was markedly increased compared to concurrently incubated PBMC (4000 ng p24/ml in MMC compared to 550 ng p24/ml in PBMC). The data, summarized in FIG. 14, support the hypothesis that the increased co-receptor expression on mucosal CD4 T cells enhances infectivity by HIV.

Analysis of CD8+ and CCR5 Cells in Colon.

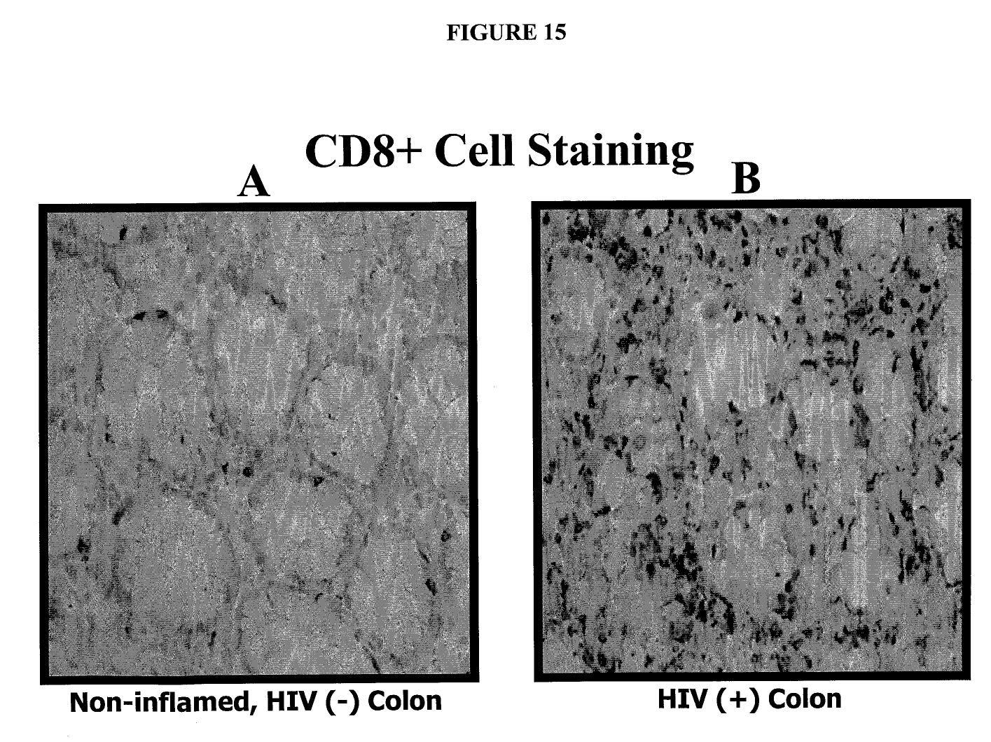

Typically in the intraepithelial compartment the majority of cells are CD8+, but the majority of lamina propria lymphocytes are CD4+. In order to determine the relative amount of CD8+ cells in colon, biopsies of colon mucosa were analyzed for the presence of CD8+ cells. The results in FIG. 15 indicate that the degree of CD8+ staining (brown cells, darkened in a black/white image) in a biopsy from an HIV-infected patient is greater than in an HIV negative biopsy (compare panels A and B). In particular, the majority of the cells in the lamina propria are CD8+ in an HIV-infected patient rather than CD4+. Furthermore, the numbers of CD4+ cells are severely depleted in colon mucosum of an HIV-infected patient. In this case, if the biopsy were examined microscopically, it would be said to be normocellular rather than inflammed due to the increase in CD8 cells being balanced by a loss of CD4 cells caused by HIV-induced CD4 cell killing. This likely accounts for why earlier observations of mucosa failed to detect inflammation in HIV affected mucosal tissues. There is actual cellular inflammation related to CD8 cells.

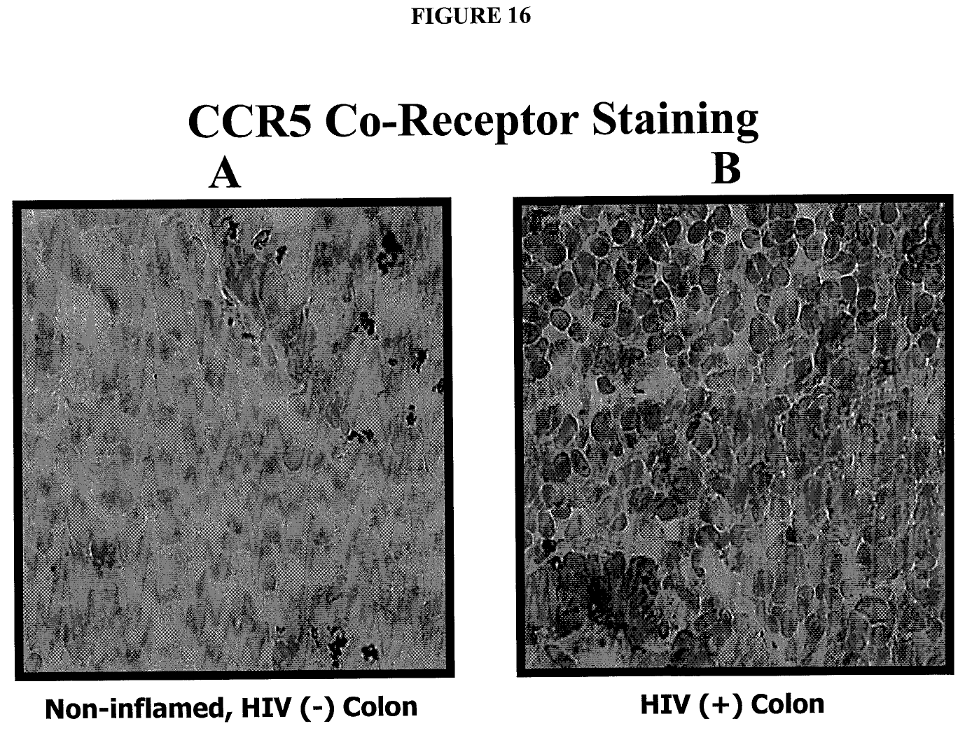

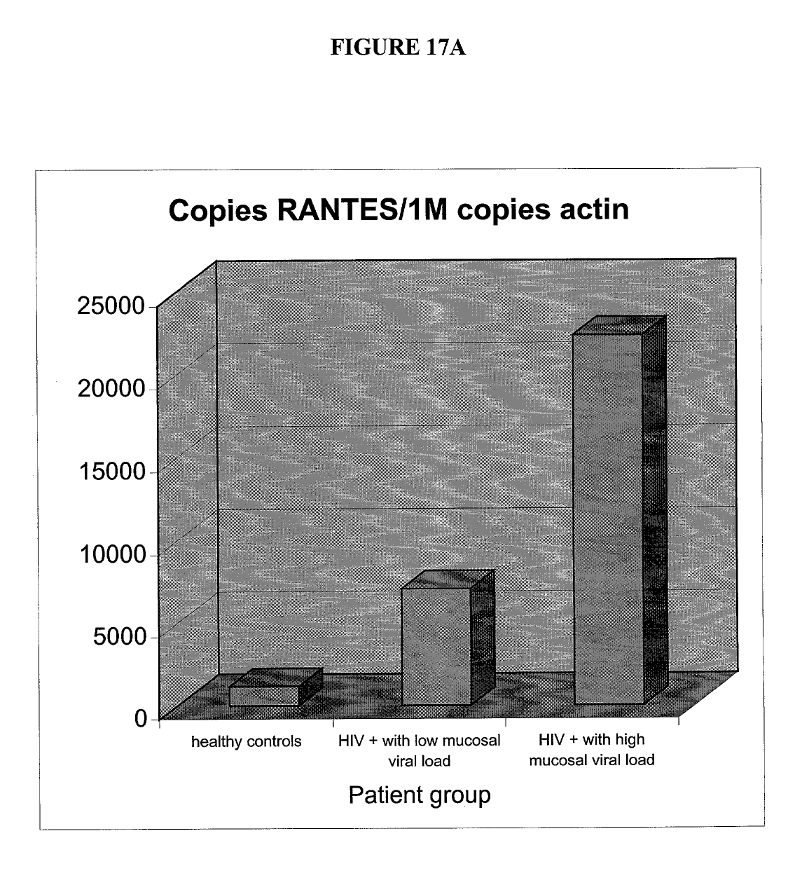

The b-chemokines discussed above, previously shown to be elevated by HIV, recruit cells that bear the CCR5 receptor to the mucosa (FIG. 16). The CCR5 receptor is the primary co-receptor that HIV uses to enter these cells. Thus HIV is ensuring that it will be able to propogate further by inducing inflammation in this environment, at least in part via production of pro-inflammatory cytokines, thereby recruiting more target cells for infection.

Quantitation of β-Chemokine Tissue Concentrations Using PCR Analysis

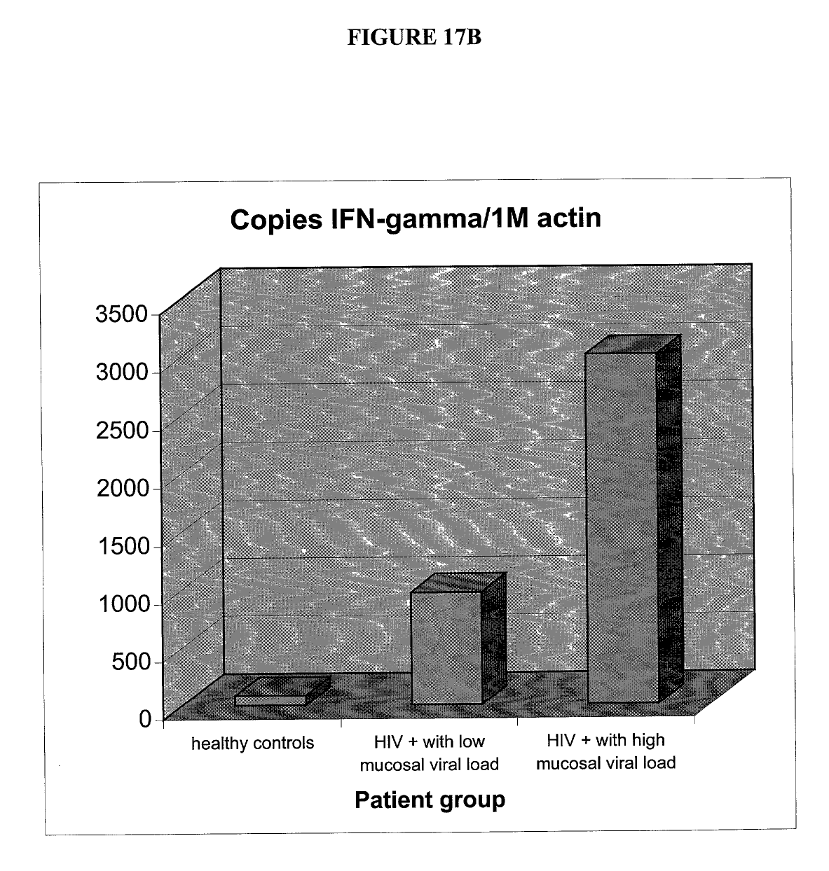

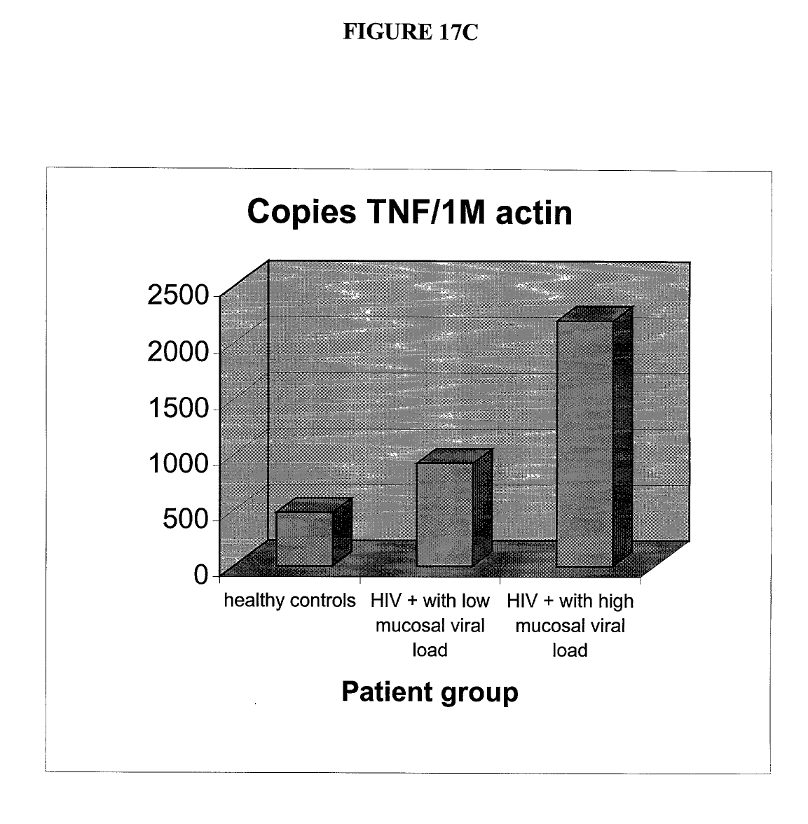

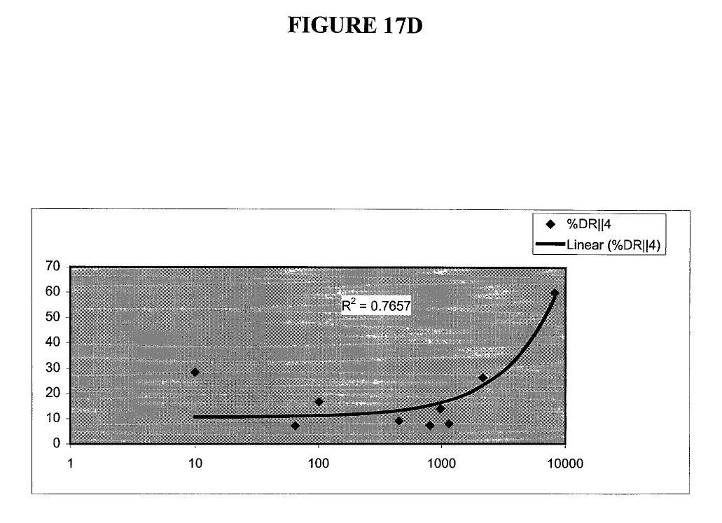

Further evidence that HIV is an inflammatory disease of the mucosa and, therefore, is amenable to treatment with anti-inflammatory agents is shown in FIGS. 17(A-C). The data show concentrations of pro-inflammatory cytokines and chemokines (mRNA), RANTES, IFNγ, and TNF (RNA), present in mucosal biopsies of normal healthy control patients, HIV patients with low amounts of virus in their mucosa and patients with higher amounts of HIV in mucosa. As can clearly be seen in these studies, patients with HIV in their mucosa have higher levels of the pro-inflammatory chemokine RANTES, interferon-gamma (IFNγ), and TNF compared to healthy patients. Furthermore, HIV patients with higher mucosal viral load have much higher levels of RANTES, IFNγ, and TNF than HIV patients with low mucosal viral load. In both cases the comparison of the HIV patients with the healthy normals was statistically significant (RANTES, p=0.0008 and 0.001, respectively; IFNγ, p=0.0008 and 0.002, respectively; and TNF, p=0.002 and 0.01, respectively).

HIV Load Increases Activation of CD4+ Cells and Production of Viral Progeny.

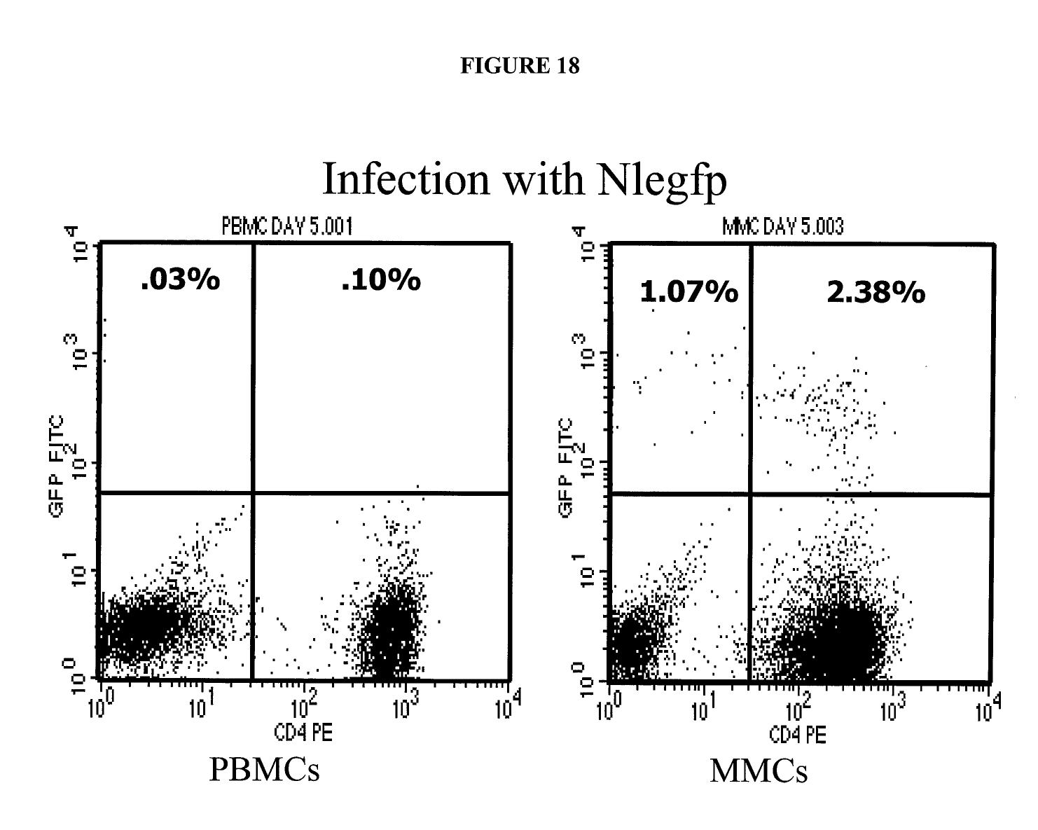

FIG. 17D shows that as mucosal viral load increases, there is an increase in the percentage of CD4+ cells in the mucosa that are activated (as indicated by increased expression of HLA-DR). As shown in FIG. 18, the mucosa cells, once infected will produce more virus. This is indicated by increased expression of green flouresence protein by a T-tropic reporter virus (Nlegfp) when replicated in a cell. The results indicate that the percentage of mucosal cells producing virus is more than 300 times greater than in blood cells.

Thus, these results demonstrate that HIV increases the levels of pro-inflammatory cytokines which leads to activation of CD4+ cells, the exact cell type that is injectable by HIV. Infected CD4+ cells in turn produce progeny virus available to infect other CD4+ cells and cells recruited by the production of the various pro-inflammatory cytokines and chemokines thereby spreading the infection.

The Anti-Inflammatory Agent 5-ASA Inhibits Viral Replication in Cells.

In order to evaluate the ability of 5-ASA to inhibit HIV replication in vitro, HIV infection studies using 1×10⁶ activated PBMCs in the presence of graded doses of 5-ASA (3, 30, 300, 3,000 μM) was performed. The PBMCs comprise a heterogeneous population of CD4 and CD8 cells with chemokines and cytokines present in the medium. 5-ASA dose ranges used are believed to represent the physiologic range seen in the gastrointestinal mucosa of patients treated with 4.8 g of oral 5-ASA per day. Replication incompetent HIV pNLlucΔBgl pseudotyped with either M-tropic JRFL or T-tropic LAI envelope was used for the infection. Luciferase expression was used to assess the degree of HIV replication after 4 days in culture. Medication treated cultures were compared with samples receiving no drug. Cells were analyzed by flow cytometry for cell death after 4 days by staining with 7-AAD.

The results in Table 2 show that 5-ASA treatment produced a 3 to 67% inhibition of luciferase expression with inhibition being significant at the highest dose (P<0.005) (N=7 infections per treatment group). Cell death did not appear to account for the observed inhibition (% death in un-treated controls=7.8%, 30 μM 5-ASA=7.3%, 300 μM 5-ASA=9.4% and 3000 μM 5-ASA=13.3%).

TABLE 2

| | % Change from | | % Change from |
|---|---|---|---|
| M-tropic HIV | untreated culture | T-Tropic HIV | untreated culture |
| ASA 3 μM | 46.2 | ASA 2 μM | 3.2 |
| ASA 30 μM | 50.3 | ASA 20 μM | 22.4 |
| ASA 300 μM | 48.9 | ASA 200 μM | 26.0 |
| ASA 3000 μM | 67.6* | ASA 2000 μM | 66.7* |
| AZT 5 μM | 80.0* | AZT 5 μM | 99.3* |

*P < .05

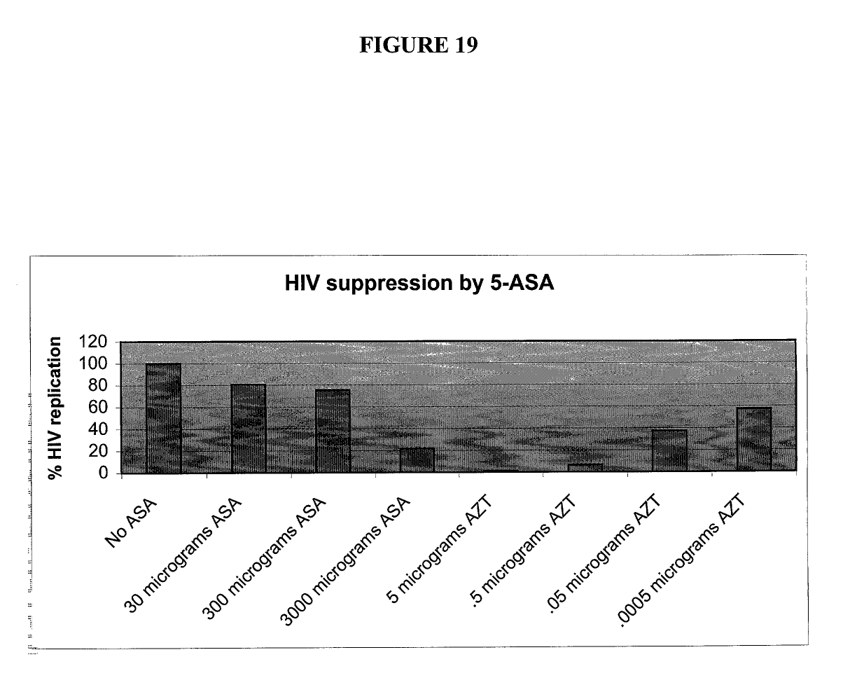

FIG. 19 shows that the use of Asacol (mesalamine), a topical anti-inflammatory agent, inhibits HIV replication. Using a culture of cells that are infected with an HIV virus that expresses luciferase when replicated, but without any treatment as a control, Asacol at the two lowest doses (30 and 300 micrograms) is able to inhibit HIV replication by approximately 20%. At the 3000 microgram dose, the amount of suppression is higher, but this is due, in part, to cellular toxicity of the drug at this dose (approximately 30% cell death vs. 13-15% cell death for 30 and 300 microgram doses; AZT produces approximately 16% cell death at a dose of 5 μg). These results are shown compared to the more effective, known direct antiviral AZT, which blocks the viruses ability to reverse transcribe its genetic material (RNA) into DNA for subsequent integration into the cell's DNA. While the anti-inflammatory activity of Asacol is not as potent at suppressing HIV replication as is AZT, the activity is nonetheless statistically significant.

In sum, the data show that 5-ASA (Asacol) in presumed physiologic concentrations inhibited HIV replication at the highest dose and that the inhibitory activity appears to be independent of drug-induced cell toxicity. The inhibitory activity of 5-ASA is likely, at least in part, due to its anti-inflammatory activity. Therefore, these results indicate the broad applicability of using anti-inflammatory agents for treating HIV.

A number of embodiments of the present invention have been described. Nevertheless, it will be understood that various modifications may be made without departing from the spirit and scope of the invention.

What is claimed is:

1. A method of inhibiting activation of a retrovirus, comprising contacting a cell infected with the virus with a virus-activation inhibiting amount of an anti-inflammatory agent and an antiviral agent.

2. The method of claim 1, wherein the retrovirus is a lentivirus.

3. The method of claim 2, wherein the lentivirus is an immunodeficiency virus.

4. The method of claim 3, wherein the immunodeficiency virus is selected from the group consisting of human immunodeficiency virus (HIV) type 1, HIV-type 2, and simian immunodeficiency virus (SIV).

5. The method of claim 1, wherein the contacting is in-vivo.

6. The method of claim 1, wherein the contacting is in vitro.

7. The method of claim 1, wherein the cell is a mammalian cell.

8. The method of claim 7, wherein the mammalian cell is a human cell.

9. The method of claim 1, wherein the anti-viral agent inhibits viral fusion or cell entry, viral reverse transcription or nucleic acid replication, viral integration into cell DNA, viral budding or release from a cell, production of infectious virus, or an enzyme associated with viral fusion or infection, reverse transcription or nucleic acid replication, viral integration into cell DNA, viral budding or release from a cell, or production of infectious virus.

10. The method of claim 1, wherein the anti-viral agent is a polypeptide or functional mimetic.

11. The method of claim 10, wherein the polypeptide or functional mimetic binds to the virus or a cell surface receptor.

12. The method of claim 10, wherein the polypeptide is a ligand, a viral receptor, an antibody or a fragment thereof.

13. The method of claim 9, wherein the enzyme is a protease, a reverse transcriptase or an integrase.

14. The method of claim 1, wherein the anti-viral agent is selected from the group consisting of a protease inhibitor, a nucleoside reverse transcriptase inhibitor, a non-nucleoside reverse transcriptase inhibitor, an integrase inhibitor and mixtures thereof.

15. The method of claim 14, wherein the nucleoside inhibitor is zidovudine (AZT), stavudine (d4T), lamivudine (3TC), didanosine (DDI), zalcitabine (ddC), abacavir and mixtures thereof.

16. The method of claim 14, wherein the non-nucleoside inhibitor is selected from the group consisting of nevirapine, delavirdine and efavirenz.

17. The method of claim 1, wherein the anti-viral agent is a protease inhibitor.

18. The method of claim 17, wherein the protease inhibitor is saquinavir, ritonavir, indinavir, nelfinavir, or amprenavir.

19. The method of claim 1, wherein the anti-inflammatory agent decreases the recruitment of inflammatory cells, decreases the production of chemokines, decreases the production of pro-inflammatory cytokines, or inhibits interaction of a chemokine receptor with its ligand.

20. The method of claim 1, wherein the anti-inflammatory agent is selected from the group consisting of an anti-inflammatory antibody, an anti-inflammatory peptide, an anti-inflammatory cytokine, an anti-inflammatory chemokine, an anti-inflammatory nucleic acid, a steroid, a non-steroidal anti-inflammatory drug, a 5-ASA product, and combinations thereof.

21. The method of claim 20, wherein the anti-inflammatory antibody is selected from the group consisting of an anti-cytokine antibody, an anti-cytokine receptor antibody, an anti-chemokine antibody, an anti-chemokine receptor antibody, an anti-proinflammatory peptide antibody, and combinations thereof.

22. The method of claim 20, wherein the anti-inflammatory peptide is selected from the group consisting of an LFA adhesion molecule antagonist, a cytokine receptor antagonist, a transcription factor, and a soluble TNF-α receptor polypeptide.

23. The method of claim 20, wherein the anti-inflammatory cytokine is selected from the group consisting of IL-4, IL-10, IL-13, IL-16, and combinations thereof.

24. The method of claim 20, wherein the anti-inflammatory nucleic acid is selected from the group consisting of a ribozyme, a nucleic acid encoding an anti-inflammatory peptide, an antisense nucleic acid, and combinations thereof.

25. The method of claim 24, wherein the antisense nucleic acid hybridizes to a nucleic acid encoding a cytokine receptor, an inflammatory cytokine, a chemokine receptor, or a chemokine.

26. The method of claim 20, wherein the steroid is a glucocorticoid.

27. The method of claim 20, wherein the steroid is selected from the group consisting of flunisolide, triamcinoline, triamcinoline acetonide, beclomethasone diproprionate, betamethasone diproprionate, hydrocortisone, cortisone, dexamethasone, budesonide, prednisone, methyl prednisolone, prednisolone, and combinations thereof.

28. The method of claim 20, wherein the non-steroidal anti-inflammatory drug is selected from the group of salicylic acid derivatives consisting of salicylic acid, sodium thiosalicylate, choline salicylate, magnesium salicylate, diflunisal, ibuprofen, naproxen, sulindac, diflunisal, salicylsalicylic acid, choline magnesium trisalicylate, acetylsalicylic acid, salsalate, sodium salicylate and combinations thereof.

29. The method of claim 20, wherein the non-steroidal anti-inflammatory drug is selected from the group consisting of flurbiprofen, fenoprofen, naburnetone, ketoprofen, piroxicam, indomethacin, tolmetin, meclofanamate sodium, mefenamic acid, etodolac, ketorolac tromethamine, diclofenac, oxaprozin, bromfenac sodium, rofecoxib, suprofen, fenbuprofen, fluprofen, thalidomide, evening primrose oil, single isomers thereof and combinations thereof.

30. The method of claim 20, wherein the 5-ASA product is selected from the group consisting of mesalamine, balsalazide, ipsalazide, olsalazine, sulfasalazine and mixtures thereof.

31. A method for inhibiting an inflammatory mediated infection of mucosal tissue, comprising contacting the tissue with an inhibiting effective amount of an anti-inflammatory agent and an antiviral agent.

32. The method of claim 31, wherein the inflammatory mediated infection is caused by a virus.

33. The method of claim 32, wherein the virus is a retrovirus.

34. The method of claim 33, wherein the retrovirus is a lentivirus.

35. The method of claim 34, wherein the lentivirus is an immunodeficiency virus.

36. The method of claim 35, wherein the immunodeficiency virus is selected from the group consisting of human immunodeficiency virus (HIV) type 1, HIV-type 2, and simian immunodeficiency virus (SIV).

37. The method of claim 31, wherein the contacting is in vivo.

38. The method of claim 31, wherein the contacting is in vitro.

39. The method of claim 31, wherein the contacting is ex vivo.

40. The method of claim 31, wherein the tissue is a mammalian tissue.

41. The method of claim 40, wherein the mammalian tissue is a human tissue.

42. The method of claim 31, wherein the mucosal tissue is a vaginal tissue, a gastro-intestinal tissue, a nasal tissue or a tissue of the lower GI tract.

43. The method of claim 31, wherein the contacting is by administering the anti-inflammatory agent locally or systemically, prior to, simultaneously with or after administering the antiviral agent.

44. The method of claim 43, wherein the administration is by locally contacting by topical administration.

45. The method of claim 43, wherein the systemic administration is by intravenous, oral or parenteral administration.

46. The method of claim 31, wherein the anti-viral agent inhibits viral fusion or cell entry, viral reverse transcription or nucleic acid replication, viral integration into cell DNA, viral budding or release from a cell, production of infectious virus, or an enzyme associated with viral fusion or infection, reverse transcription or nucleic acid replication, viral integration into cell DNA, viral budding or release from a cell, or production of infectious virus.

47. The method of claim 31, wherein the anti-viral agent is a polypeptide or functional mimetic.

48. The method of claim 47, wherein the polypeptide or functional mimetic binds to the virus or a cell surface receptor.

49. The method of claim 47, wherein the polypeptide is a ligand, a viral receptor, an antibody or a fragment thereof.

50. The method of claim 46, wherein the enzyme is a protease, a reverse transcriptase or an integrase.

51. The method of claim 31, wherein the anti-viral agent is selected from the group consisting of a protease inhibitor, a nucleoside reverse transcriptase inhibitor, a non-nucleoside reverse transcriptase inhibitor, an integrase inhibitor and mixtures thereof.

52. The method of claim 51, wherein the nucleoside inhibitor is zidovudine (AZT), stavudine (d4T), larnivudine (3TC), didanosine (DDI), zalcitabine (ddC), abacavir and mixtures thereof.

53. The method of claim 51, wherein the non-nucleoside inhibitor is nevirapine, delavirdine, or efavirenz.

54. The method of claim 31, wherein the anti-viral agent is a protease inhibitor, a reverse transcriptase inhibitor or an integrase inhibitor.

55. The method of claim 54, wherein the protease inhibitor is saquinavir, ritonavir, indinavir, nelfinavir, or amprenavir.

56. The method of claim 31, wherein the anti-inflammatory agent decreases the recruitment of inflammatory cells, decreases the production of chemokines, decreases the production of pro-inflammatory cytokines, or inhibits the interaction of a chemokine receptor with its ligand.

57. The method of claim 31, wherein the anti-inflammatory agent is selected from the group consisting of an anti-inflammatory antibody, an anti-inflammatory peptide, an anti-inflammatory cytokine, an anti-inflammatory chemokine, an anti-inflammatory nucleic acid, a steroid, a non-steroidal anti-inflammatory drug, 5-ASA products, and combinations thereof.

58. The method of claim 57, wherein the anti-inflammatory antibody is selected from the group consisting of an anti-cytokine antibody, an anti-cytokine receptor antibody, an anti-chemokine antibody, an anti-chemokine receptor antibody, an anti-proinflammatory peptide antibody, and combinations thereof.

59. The method of claim 57, wherein the anti-inflammatory peptide is selected from the group consisting of an LFA-1 antagonist, a cytokine receptor antagonist, a transcription factor, and a soluble TNF-α receptor.

60. The method of claim 57, wherein the anti-inflammatory cytokine is selected from the group consisting of IL-4, IL-10, IL-13, IL-16, and combinations thereof.

61. The method of claim 57, wherein the anti-inflammatory nucleic acid is selected from the group consisting of a ribozyme, a nucleic acid encoding an anti-inflammatory peptide, an antisense nucleic acid, and combinations thereof.

62. The method of claim 61, wherein the antisense nucleic acid hybridizes to nucleic acid encoding a cytokine receptor, an inflammatory cytokine, a chemokine, or a chemokine receptor.

63. The method of claim 57, wherein the steroid is a glucocorticoid.

64. The method of claim 57, wherein the steroid is selected from the group consisting of flunisolide, triamcinoline, triamcinoline acetonide, beclomethasone diproprionate, betamethasone diproprionate, hydrocortisone, cortisone, dexamethasone, budesonide, prednisone, methyl prednisolone, prednisolone, and combinations thereof.

65. The method of claim 57, wherein the non-steroidal anti-inflammatory drug is selected from the group of salicylic acid derivatives consisting of salicylic acid, sodium thiosalicylate, choline salicylate, magnesium salicylate, diflunisal, ibuprofen, naproxen, sulindac, diflunisal, salicylsalicylic acid, choline magnesium trisalicylate, acetylsalicylic acid, salsalate, sodium salicylate and combinations thereof.

66. The method of claim 57, wherein the non-steroidal anti-inflammatory drug is selected from the group consisting of flurbiprofen, fenoprofen, naburnetone, ketoprofen, piroxicam, indomethacin, tolnetin, meclofanamate sodium, mefenamic acid, etodolac, ketorolac tromethamine, diclofenac, oxaprozin, bromfenac sodium, rofecoxib, suprofen, fenbuprofen, fluprofen, thalidomide, evening primrose oil, single isomers thereof and combinations thereof.

67. The method of claim 57, wherein the 5-ASA product is selected from the group consisting of mesalamine, balsalazide, ipsalazide, olsalazine, sulfasalazine and mixtures thereof.

68. A method of decreasing the probability of an inflammatory mediated mucosal infection in a subject at risk of having an inflammatory mediated mucosal infection, comprising contacting the subject with an effective amount of an anti-inflammatory agent and an antiviral agent.

69. The method of claim 68, wherein the inflammatory mediated mucosal infection is caused by a virus.

70. The method of claim 69, wherein the virus is a retrovirus.

71. The method of claim 70, wherein the retrovirus is a lentivirus.

72. The method of claim 71, wherein the lentivirus is an immunodeficiency virus.

73. The method of claim 70, wherein the immunodeficiency virus is selected from the group consisting of human immunodeficiency virus (HIV) type 1, HIV-type 2, and simian immunodeficiency virus (SIV).

74. The method of claim 68, wherein the contacting is in vivo.

75. The method of claim 68, wherein the contacting in vivo is by administering the anti-inflammatory agent locally or systemically, prior to, simultaneously with or after administering the antiviral agent.

76. The method of claim 75, wherein the administration is by locally contacting by topical administration.

77. The method of claim 75, wherein the systemic contacting is by intravenous, oral or parenteral administration.

78. The method of claim 68, wherein the subject is a mammal.

79. The method of claim 78, wherein the mammal is a human.

80. The method of claim 68, wherein the anti-viral agent inhibits viral fusion or cell entry, viral reverse transcription or nucleic acid replication, viral integration into cell DNA, viral budding or release from a cell, production of infectious virus, or an enzyme associated with viral fusion or infection, reverse transcription or nucleic acid replication, viral integration into cell DNA, viral budding or release from a cell, or production of infectious virus.

81. The method of claim 68, wherein the anti-viral agent is a polypeptide or functional mimetic.

82. The method of claim 81 wherein the polypeptide or functional mimetic binds to the virus or a cell surface receptor.

83. The method of claim 81, wherein the polypeptide is a ligand, a viral receptor, an antibody or a fragment thereof.

84. The method of claim 80, wherein the enzyme is a protease, a reverse transcriptase or an integrase.

85. The method of claim 68, wherein the anti-viral agent is selected from the group consisting of a protease inhibitor, a nucleoside reverse transcriptase inhibitor, a non-nucleoside reverse transcriptase inhibitor, an integrase inhibitor and mixtures thereof.

86. The method of claim 85, wherein the nucleoside inhibitor is zidovudine (AZT), stavudine (d4T), laruivudine (3TC), didanosine (DDI), zalcitabine (ddC), abacavir and mixtures thereof.

87. The method of claim 85, wherein the non-nucleoside inhibitor is nevirapine, delavirdine, or efavirenz.

88. The method of claim 68, wherein the anti-viral agent is a protease inhibitor, a reverse transcriptase inhibitor or an integrase inhibitor.

89. The method of claim 88, wherein the protease inhibitor is saquinavir, ritonavir, indinavir, nelfinavir, or amprenavir.

90. The method of claim 68, wherein the anti-inflammatory agent causes a decrease in the recruitment of inflammatory cells, a decrease in the production of chemokines, a decrease in the production of pro-inflammatory cytokines, or inhibit interaction of a chemokine receptor with its ligand.

91. The method of claim 68, wherein the anti-inflammatory agent is selected from the group consisting of an anti-inflammatory antibody, an anti-inflammatory peptide, an anti-inflammatory cytokine, an anti-inflammatory chemokine, an anti-inflammatory nucleic acid, a steroid, a non-steroidal anti-inflammatory drug, 5-ASA products, and combinations thereof.

92. The method of claim 91, wherein the anti-inflammatory antibody is selected from the group consisting of an anti-cytokine antibody, an anti-cytokine receptor antibody, an anti-chemokine antibody, an anti-chemokine receptor antibody, an anti-proinflammatory peptide antibody, and combinations thereof.

93. The method of claim 91, wherein the anti-inflammatory peptide is selected from the group consisting of an LFA-1 antagonist, a cytokine receptor antagonist, a transcription factor, and a soluble TNF-α receptor.

94. The method of claim 91, wherein the anti-inflammatory cytokine is selected from the group consisting of IL-4, IL-10, IL-13, IL-16, and combinations thereof.

95. The method of claim 91, wherein the anti-inflammatory nucleic acid is selected from the group consisting of a ribozyme, a nucleic acid encoding an anti-inflammatory peptide, an antisense nucleic acid, and combinations thereof.

96. The method of claim 95, wherein the antisense nucleic acid hybridizes to a nucleic acid encoding a cytokine receptor, inflammatory cytokine, a chemokine, or a chemokine receptor.

97. The method of claim 91, wherein the steroid is selected from the group consisting of flunisolide, triamcinoline, triamcinoline acetonide, beclomethasone diproprionate, betamethasone diproprionate, hydrocortisone, cortisone, dexamethasone, budesonide, prednisone, methyl prednisolone, prednisolone, and combinations thereof.

98. The method of claim 91, wherein the non-steroidal anti-inflammatory drug is selected from the group of salicylic acid derivatives consisting of salicylic acid, sodium thiosalicylate, choline salicylate, magnesium salicylate, diflunisal, ibuprofen, naproxen, sulindac, diflunisal, salicylsalicylic acid, choline magnesium trisalicylate, acetylsalicylic acid, salsalate, sodium salicylate and combinations thereof.

99. The method of claim 91, wherein the non-steroidal anti-inflammatory drug is selected from the group consisting of flurbiprofen, fenoprofen, naburnetone, ketoprofen, piroxicam, indomethacin, tolmetin, meclofanamate sodium, mefenamic acid, etodolac, ketorolac tromethamine, diclofenac, oxaprozin, bromfenac sodium, rofecoxib, suprofen, fenbuprofen, fluprofen, thalidomide, evening primrose oil, single isomers thereof and combinations thereof.

100. The method of claim 91, wherein the 5-ASA product is selected from the group consisting of mesalamine, balsalazide, ipsalazide, olsalazine, sulfasalazine and mixtures thereof.

101. A method of inhibiting activation of a retrovirus, comprising contacting a cell infected with the virus with an activation-inhibiting amount of an anti-inflammatory agent, wherein the anti-inflammatory agent is distinct from 5-ASA or ASA.

102. The method of claim 101, wherein the anti-inflammatory agent is selected from the group consisting of an anti-inflammatory antibody, an anti-inflammatory peptide, an anti-inflammatory cytokine, an anti-inflammatory chemokine, an anti-inflammatory nucleic acid, a steroid, a non-steroidal anti-inflammatory drug, and combinations thereof.

103. A method for inhibiting an inflammatory mediated infection of mucosal tissue, comprising contacting the tissue with an inhibiting effective amount of an anti-inflammatory agent, wherein the anti-inflammatory agent is distinct from 5-ASA or ASA.

104. The method of claim 103, wherein the anti-inflammatory agent is selected from the group consisting of an anti-inflammatory antibody, an anti-inflammatory peptide, an anti-inflammatory cytokine, an anti-inflammatory chemokine, an anti-inflammatory nucleic acid, a steroid, a non-steroidal anti-inflammatory drug, and combinations thereof.

105. A method of inhibiting an inflammatory mediated mucosal infection in a subject having or at risk of having an inflammatory mediated mucosal infection, comprising contacting the subject with an effective amount of an anti-inflammatory agent, wherein the anti-inflammatory agent is distinct from 5-ASA or ASA.

106. The method of claim 105, wherein the anti-inflammatory agent is selected from the group consisting of an anti-inflammatory antibody, an anti-inflammatory peptide, an anti-inflammatory cytokine, an anti-inflammatory chemokine, an anti-inflammatory nucleic acid, a steroid, a non-steroidal anti-inflammatory drug, and combinations thereof.

107. A method of inhibiting progression of an HIV-related disorder in a subject having a human immunodeficiency virus, comprising administering to the subject having the HIV viral infection a therapeutically effective amount of an anti-inflammatory agent that inhibits progression of the HIV virus.

108. The method of claim 107, wherein the anti-inflammatory agent is distinct from ASA or 5-ASA.

109. The method of claim 107, further comprising administering an antiviral agent.

110. A method for preventing or decreasing the probability of infection of a subject at risk of an HIV infection, comprising administering to the subject a prophylactic effective amount of an anti-inflammatory agent which inhibits or decreases the probability of HIV infection of the subject.

111. The method of claim 110, wherein the anti-inflammatory agent is distinct from ASA or 5-ASA.

112. The method of claim 110, further comprising administering an antiviral agent, prior to, simultaneously with or after administering the anti-inflammatory agent.

113. The method of claims 107 or 110, wherein the anti-inflammatory agent is administered topically.

114. The method of claims 107 or 110, wherein the anti-inflammatory agent is administered systemically.

115. A pharmaceutical composition comprising at least one dose of a therapeutically effective amount of an anti-inflammatory agent, in a pharmaceutically acceptable carrier, wherein the dose is in an amount effective to inhibit or decrease the probability of immunodeficiency virus infection.

116. The pharmaceutical composition of claim 115, further comprising an antiviral agent.

117. The pharmaceutical composition of claim 115, further comprising a pharmaceutically acceptable gel, cream, foam or suppository.

118. An article of manufacture, comprising at least one anti-inflammatory agent and instructions for use of the agent in treating, preventing or decreasing the probability of an immunodeficiency virus infection.

119. The article of manufacture of claim 118, further comprising an antiviral agent.

120. The article of manufacture of claim 118, wherein the article is selected from the group consisting of a condom, a sponge, a diaphragm, a cervical cap, a vaginal ring, a suppository, and an enema.

121. A method of inhibiting activation of a retrovirus in a tissue infected with the virus, comprising contacting the tissue with an activation-inhibiting effective amount of an anti-inflammatory agent.

122. The method of claim 121, wherein the anti-inflammatory agent is distinct from ASA or 5-ASA.

123. The method of claim 121, further comprising contacting the tissue with an antiviral agent prior to, simultaneously with or after administering the anti-inflammatory agent.

124. A method for inhibiting activation of a retrovirus in a subject infected with the virus, comprising contacting the subject with an activation-inhibiting amount of an anti-inflammatory agent.

125. The method of claim 124, wherein the anti-inflammatory agent is distinct from ASA or 5-ASA.

126. The method of claim 124, further comprising contacting the subject with an antiviral agent prior to, simultaneously with or after administering the anti-inflammatory agent.

127. An article of manufacture, comprising at least one anti-inflammatory agent and instructions for use of the agent in prophylaxis of immunodeficiency virus infection.

128. The article of manufacture of claim 127, further comprising an antiviral agent.

129. The article of manufacture of claim 127, wherein the article is selected from the group consisting of a condom, a sponge, a diaphragm, a cervical cap, a vaginal ring, a suppository, and an enema.
